(19) United States
(12) Patent Application Publication
(10) Pub. No.: US 2004/0229778 A1
(43) Pub. Date: Nov. 18, 2004

(54) Pharmaceutical compositions of antithrombin III for the treatment of retroviral diseases

(76) Inventors: David R. Elmaleh, Newton, MA (US); Ralf Geiben Lynn, Watertown, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/436,872

(22) Filed: May 13, 2003

(51) Int. Cl. A61K 38/39; A61K 31/715; A61K 38/48

(52) U.S. Cl. 514/12; 424/94.64; 514/54

(57) ABSTRACT

Pharmaceutical compositions comprised of high molecular weight ATIII are discloses, as is the use thereof in treating infectious diseases, inflammatory disorders and diseases or conditions that are mediated by thrombin activation.

### BACKGROUND

Retroviral Diseases

The human retrovirus, human immunodeficiency virus (HIV) causes Acquired Immunodeficiency Syndrome (AIDS), an incurable disease in which the body's immune system breaks down leaving the victim vulnerable to opportunistic infections, e.g., pneumonia, and certain cancers, e.g., Karposis Sarcoma. AIDS is a global health problem. The Joint United Nations Programme on HIV/AIDS (UNAIDS) estimates that there are now over 34 million people living with HIV or AIDS worldwide. Some 28.1 million of those infected individuals reside in impoverished sub-Saharan Africa. In the United States, one out of every 250 people are infected with HIV or have AIDS. Since the beginning of the epidemic, AIDS has killed nearly 19 million people worldwide, including an estimated 425,000 Americans. AIDS has replaced malaria and tuberculosis as the world's deadliest infectious disease among adults and is the fourth leading cause of death worldwide.

There is still no cure for AIDS. There are, however, a variety of antiretroviral drugs that prevent HIV from reproducing and ravaging the body's immune system. One such class of drugs are the reverse transcriptase inhibitors which attack an HIV enzyme called reverse transcriptase. Another class of drugs is the protease inhibitors which inhibit HIV enzyme protease. First introduced in 1995, these protease inhibitors are now widely used for the treatment of HIV infection alone or in combination with other antiretroviral drugs. Today, approximately 215,000 of the estimated 350,000 patients receiving treatment for HIV infection in the United States are treated with at least one drug from the protease inhibitor class of drugs.

Highly active antiretroviral drug therapy (HAART) is a widely used anti-HIV therapy that entails triple-drug protease inhibitor-containing regimens that can completely suppress viral replication (Stephenson, JAMA, 277: 614-6 (1997)). The persistence of latent HIV in the body, however, has been underestimated. It is now recognized that there exists a reservoir of HIV in perhaps tens of thousands to a million long-lived resting “memory” T lymphocytes (CD4), in which the HIV genome is integrated into the cells own DNA (Stephenson, JAMA, 279: 641-2 (1998)). This pool of latently infected cells is likely established during primary infection.

Such combination therapy is often only partially effective, and it is unknown how much viral suppression is required to achieve durable virologic, immunologic, and clinical benefit (Deeks, JAMA, 286: 224-6 (2001)). Anti-HIV drugs are highly toxic and can cause serious side effects, including heart damage, kidney failure, and osteoporosis. Long-term use of protease inhibitors has been linked to peripheral wasting accompanied by abnormal deposits of body fat. Other manifestations of metabolic disruptions associated with protease inhibitors include increased levels of triglycerides and cholesterol, pancreatitis, atherosclerosis, and insulin resistance (Carr et al., Lancet, 351: 1881-3 (1998)). The efficacy of current anti-HIV therapy is further limited by the complexity of regimens, pill burden, and drug-drug interactions. Compliance with the toxic effects of antiretroviral drugs make a lifetime of combination therapy a difficult prospect and many patients cannot tolerate long-term treatment with HAART. There is an urgent need for other antiviral therapies due to poor adherence to combination therapy regimes, which has led to the emergence of drug-resistant strains of HIV. Other drugs may improve compliance by substantially reducing the daily “pill burden” and simplifying the complicated dietary guidelines associated with the use of current protease inhibitors.

The HIV virus enters the body of an infected individual and lives and replicates primarily in the white blood cells. The hallmark of HIV infection, therefore, is a decrease in cells called T-helper or CD4 cells of the immune system. The molecular mechanism of HIV entry into cells involves specific interactions between the viral envelope glycoproteins (env) and two target cell proteins, CD4 and a chemokine receptor. HIV cell tropism is determined by the specificity of the env for a particular chemokine receptor (Steinberger et al., Proc. Natl. Acad. Sci. USA. 97: 805-10 (2000)). T-cell-line-tropic (T-tropic) viruses (X4 viruses) require the chemokine receptor CXR4 for entry. Macrophage (M)-tropic viruses (R5 viruses) use CCR5 for entry (Berger et al., Nature, 391: 240 (1998)). T-tropism is linked to various aspects of AIDS, including AIDS dementia, and may be important in disseminating the virus throughout the body and serving as a reservoir of virus in the body.

Furthermore, about 40% of patients with HIV are co-infected with Hepatitis C Virus (HCV). Super-infection also occurs when patients with one type of viral hepatitis become infected with a different type of hepatitis virus at a later stage. Under these conditions, the clinical symptoms and disease courses are usually more complex and serious than that of a single viral infection occurrence. Additionally, hepatic injury is a major concern as a result of antiretroviral therapy (HAART) and has been shown to occur with all classes of antiretroviral therapy. Prior studies have indicated that HCV or HBV infection may increase the likelihood of HAART-related hepatotoxicity. Therefore it is necessary to formulate a safer treatment for HIV, HAV, HBV and HCV infections.

About 200,000 Americans and 10 million people worldwide contract Hepatitis A annually. Hepatitis B is the 9th leading cause of death worldwide and there are more than 300. million chronic HBV carriers worldwide. It affects 15-20% of the population in Asia. In United State it affects only 0.1% or 1.2 million people. Hepatitis B is transmitted by human body fluids such as blood, seminal fluid, vaginal secretions, breast milk, tears, saliva, and open sores. Its methods of transmission include mother to baby, during sexual contact, deep kissing, and through the use of improper injection techniques. HBV is 100 times more infectious than HIV. What is of an increasing concern is that people who are already chronically infected with hepatitis B or C face a higher risk of dying if they additionally contract hepatitis A. Infection with one hepatitis virus type offers no immunity from infection by another.

HBV is preventable with a vaccine. Nonetheless, more than two billion individuals today have been infected at some time in their lives with HBV, and approximately 350 million are chronically infected carriers of this virus. HBV is one of the most common human pathogens, and it is the most prevalent chronic virus infection worldwide. An estimated 140,000 Americans are infected each year with hepatitis B. Approximately one to one and a quarter million Americans are chronically infected and are considered to be carriers of the hepatitis B virus. Carriers of HBV are at high risk of serious illness and death from cirrhosis of the liver and primary liver cancer, diseases that kill more than one million carriers per year. In addition, these carriers constitute a reservoir of infected individuals who perpetuate the infection from generation to generation. A carrier is infectious and can transmit hepatitis B even though he/she has no signs or symptoms.

In addition, as of September 2000, it is estimated that 5 million Americans (some estimates go as high as 15 million) are infected with hepatitis C; and up to 230,000 new hepatitis C infections occur in the U.S. every year. About 8,000 to 10,000 Americans die of HCV annually, and the toll is expected to triple in the next decade or two. It is estimated that there are over 200 million people in the world chronically infected with hepatitis C, and this large reservoir of infected persons constitutes a daunting source of potential new infections. HCV infection is the most common type of chronic viral hepatitis in the developed world. People who are already infected with HCV can get re-infected with different sub-strains of HCV. Over the next 10-20 years, chronic hepatitis B and C will become a major burden on the health care systems as patients who are currently asymptomatic with relatively mild disease symptons progress to end-stage liver disease.

Another single-stranded RNA virus is the coronavirus, a genus in the family Coronavirirdae. These large, enveloped, plus-stranded RNA viruses (27-31 kb) are prevalent pathogens of humans and domestic animals. Coronaviruses have the largest genome of all RNA viruses and replicate by a unique mechanism which results in a high frequency of recombination. The newly found Severe Acute Respiratory Syndrome (SARS) causing virus is a member of this family. It emerged in November 2002 and as of April 2003, 3,293 people in 22 countries have become ill from the infection and hundreds more have died.

There is clearly an need for new antiretroviral agents.

Antithrombin III (ATIII) Serine protease inhibitors (serpins) constitute a superfamily of structurally related proteins found in eukaryotes, including humans (Wright, BIOASSAY, 18: 453-64 (1996); Skinner et al., J. Mol. Biol. 283: 9-14 (1998); Huntington et al., J. Mol. Biol. 293: 449-55 (1999)). Included in this class is ATIII, protein C-inhibitor, activated protein C, plasminogen activator inhibitor, and alpha-1 antitrypsin.

ATIII is a glycoprotein present in blood plasma with a well-defined role in blood clotting. Specifically ATIII is a potent inhibitor of the reactions of the coagulation cascade with an apparent molecular weight of between 54 k Da and 65 kDa (Rosenberg and Damus, J. Biol. Chem. 248: 6490-505 (1973); Nordenman et al., Eur. J. Biochem., 78: 195-204 (1977); Kurachi et al., Biochemistry 15: 373-7 (1976)) of which some ten percent is contributed by four glucosamine-base carbohydrate chains (Kurachi et al., Biochemistry 15: 373-7 (1976); Petersen et al., in The Physiological Inhibitors of Coagulation and Fibrinolysis (Collen et al., eds) Elsevier, Amsterdam, p. 48 (1979)). Although the name, ATIII, implies that it works only on thrombin, it actually serves to inhibit virtually all of the coagulation enzymes to at least some extent. The primary enzymes it inhibits are factor Xa, factor IXa, and thrombin (factor IIa). It also has inhibitory actions on factor XIIa, factor Xia and the complex of factor VIIa and tissue factor but not factor VIIa and activated protein C. ATIII also inhibits trypsin, plasmin and kallikrein (Charlotte and Church, Seminars in Hematology 28:3-9 (1995). Its ability to limit coagulation through multiple interactions makes it one of the primary natural anticoagulant proteins.

ATIII acts as a relatively inefficient inhibitor on its own. However, ATIII can be activated by a simple template mechanism, or by an allosteric conformational change brought about by heparin binding (Skinner et al., J. Mol. Biol. 283: 9-14 (1998); Huntington et al., J. Mol. Biol., 293: 449-55 (1999); Belar et al., J. Mol. Biol. Chem., 275: 8733-41 (2000)). When ATIII binds heparin, the speed with which the reaction that causes inhibition occurs is greatly accelerated. This interaction is the basis of heparin based anticoagulation therapies.

U.S. Published patent application 20020127698, entitled Serpin Drugs for Treatment of HIV Infection and Method of Use Thereof, describes methods of inhibiting the infectivity of HIV with serpins, such as antithrombin III (ATIII). The patent application teaches use of ATIII in the relaxed (R), stressed (S), modified (M) or prelatent form. Modified ATIII is described as having been treated with elastase, other proteases, chemical treatment or enzymatic digestion.

International Patent Application WO 00/52034, entitled Inhibitors of Serine Protease Activity, Methods and Compositions for Treatment for Viral Infection and U.S. Pat. No. 5,532,215 also generally teach the use of serpins, including ATIII, as anti-HIV agents.

International Patent Application WO 02/22150, entitled Medicament Containing Activated Antithrombin III, teaches that it is possible to produce activated ATIII (termed “Immune Defense Activated ATIII” or “IDAAT”) in vitro through oxidation, treatment with urea and guanidine hydrochloride, proteolytic digestion, heating to 60° C., decreasing the pH to 4.0 or addition of an ATIII peptide with a sequence SEAAAS. IDAAT is reported to be a polymer of ATIII that can be used against HIV, parasites like Plasmodium falciparum and Pneumocystis carinii and bacteria like Staphylococcus aureus.

Although ATIII and other serpins have been suggested to have anti-HIV activity, the results of studies disclosed herein indicate that pure plasma derived or recombinant ATIII are inactive in lowering HIV viral loads.

### SUMMARY OF THE INVENTION

The present invention is based on the surprising finding that ATIII, that has been treated to have a higher molecular weight, effectively reduces HIV viral loads in infected cells. Based on this finding, the invention features pharmaceutical compositions comprising: a pharmaceutically acceptable carrier and a high molecular weight antithrombin III (ATIII) in an amount effective to treat a retroviral infection.

Preferred high molecular weight ATIII molecules weigh over 60 kD and are preferably in the range of about 60 kilodaltons (kD) to about 550 kD. Particularly preferred high molecular weight ATIIIs have been heat treated and/or associated with an oligosugar. Preferred oligosugars include monosaccharides, polysaccharides, heparin (low or high molecular weight and unfractionated), pectin and amino glycosides. In other preferred embodiments, the oligosugar is itself derivatized with a small molecule, for example biotin. Particularly preferred pharmaceutical preparations of high molecular weight ATIII are prepared as controlled release formulations.

In another aspect, the invention features methods of treating infection and/or inflammation based on administration of the pharmaceutical compositions of the invention. In preferred embodiments, the infection is caused by a bacteria or virus. In particularly preferred embodiments, the virus is a retrovirus. Particularly preferred retroviruses are selected from the group consisting of: HIV, HAV, HBV, HCV, CMV and SARS.

Because high molecular weight ATIII appears to work via mechanisms distinct from existing antiviral therapeutics, pharmaceutical compositions of high molecular weight ATIII may be administered in combination with other anti-viral drugs. Preferred anti-virals include reverse transcriptase inhibitors, including cocktails, such as highly active antiretroviral drug therapy (HAART) regimen (zidovudine, zalcitabine, didanosine, stavudine, lamivudine, abacavir, tenofovir, nevirapine, efavirenz, delavirdine) and protease inhibitors (saquinavir, ritonavir, indinavir, nelfinavir, amprenavir, lopinavir), adenine arabinoside, adenine arabinoside 5′-monophosphate, acyclovir, ganciclovir, famciclovir, lamivudine, clevudine, afedovir dipivoxil, entecavir, IFN-α-2b, IFN-α-2a, lymphoblastoid IFN, consensus-IFN, IFN-β, IFN-γ, pegylated IFN-α-2a, corticosteroids, or thymosin a1, IL-2, IL-12, ribavirin, cyclosporin or granulocyte macrophage colony stimulating factor.

In yet another aspect, the invention features methods of treating a subject for a disease which is caused or contributed to by thrombin activation, by administering to the subject a high molecular weight ATIII of the invention. Diseases which are caused by or contributed to by thrombin activation include sepsis, trauma, acute respiratory distress syndrome, thrombosis, stroke, restenosis, reocclusion and restenosis in percutaneous transluminal coronary angioplasty; thrombosis associated with surgery, ischemia/reperfusion injury; coagulation abnormalities in cancer or surgical patients, an antithrombin III deficiency, venous and arterial thrombosis, disseminated intravascular coagulation, microangiopathic hemolytic anemias and veno-occlusive disease (VOD).

Other features and advantages of the invention will become apparent from the following detailed description and claims.

### BRIEF DESCRIPTION OF THE DRAWINGS

FIG. 1 shows the nucleic acid and amino acid sequences of human antithrombin III (hATIII) (Seq. Id. nos. 1 and 2).

FIG. 2 is a schematic view of antithrombin III illustrating exemplary positions of residues that are involved in heparin interactions and thrombin inhibition (Pratt et al., Seminars in Hematology. 28:3-9 (1991)).

FIG. 3 is a schematic of the crystal structure of ATIII showing heparin binding sites (Skinner et al., J. Mol. Biol. 266:601-609 (1997)).

FIG. 4 (a)-(d) are a series of graphs showing inhibition of HIV-1 with various super ATIIIs as measured by the HIV-1 p24 enzyme linked immunosorbent assay (ELISA). (a) Form 1 is treated @60° C. for 24 hours; (b) Form 2 is Form 1 modified with low molecular weight heparin. (c) Form 3 is low molecular weight heparin modified ATIII. (d) Form 4 is Form 3 treated @60° C. for 24 hours.

FIG. 5(a) is a graph showing inhibition of HIV-1 as measured by the HIV-1 p24 ELISA by modified recombinant ATIII prepared by Genzyme Transgenic Corporation Biotherapeutics (GTCB) Biotherapeutics.

FIG. 5(b) shows inhibition of HIV-1 as measured by the HIV-1 ELISA for ATIIIs prepared by Calbiochem, Sigma, Roche, Form 3 and GTCB.

FIG. 6 (a-g) are High Performance Liquid Chromatography (HPLC) chromatograms of variously treated GTCB-ATIII by ultraviolet (UV) or refractive index (RI) detection. (a) GTC-ATIII (UV analysis); (b) GTC-ATIII (RI analysis); (c) GTC-ATIII heparin treated (UV analysis); (d) GTC-ATIII heparin treated (RI analysis); (e) GTC-ATIII heparin+heat treated (UV analysis); (f) GTC-ATIII heparin+heat treated (RI analysis); (g) GTC-ATIII heparin+heat treated (UV+RI analysis).

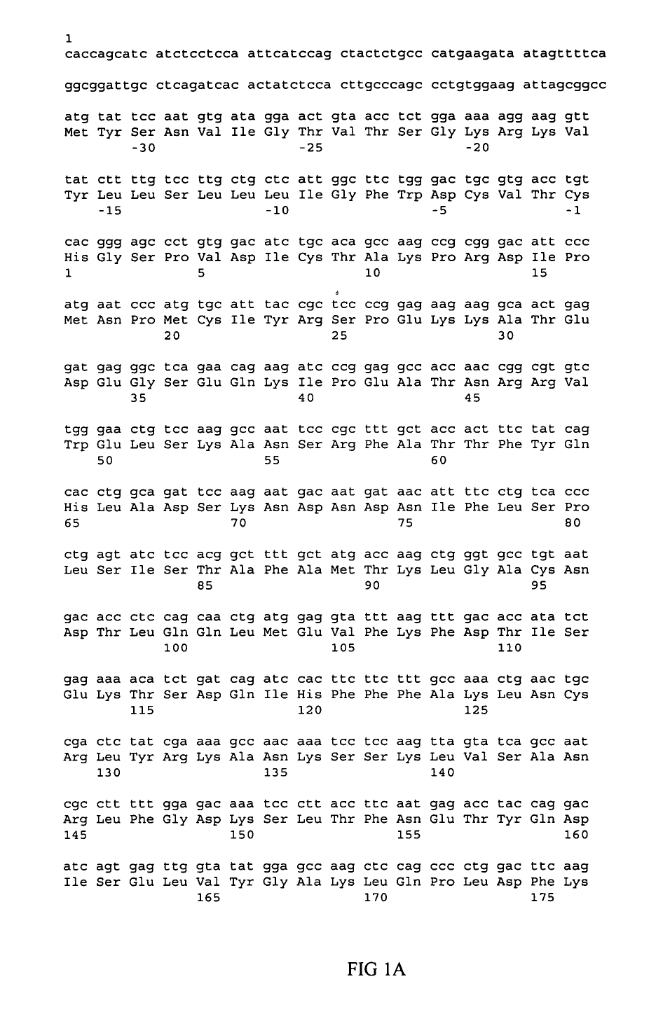

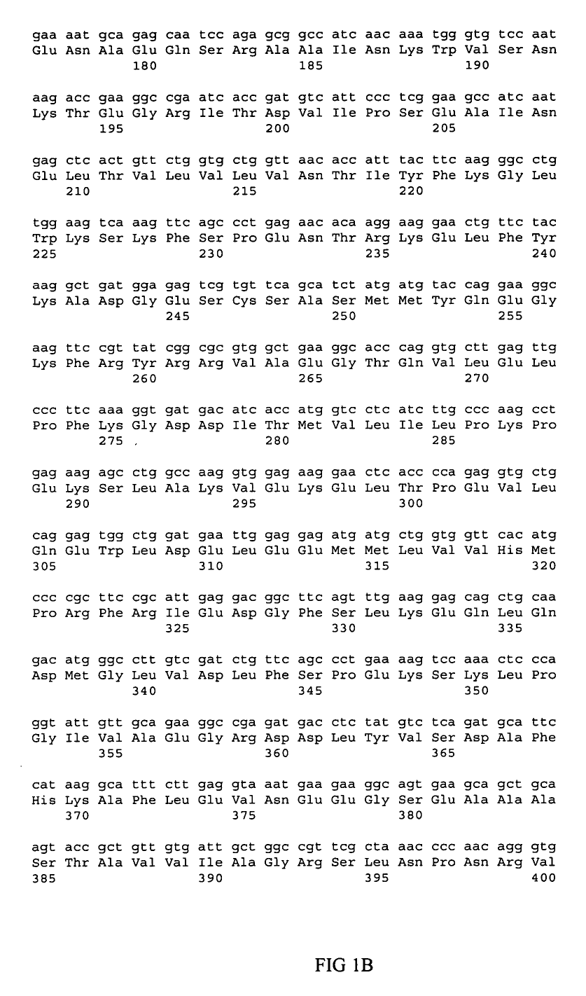

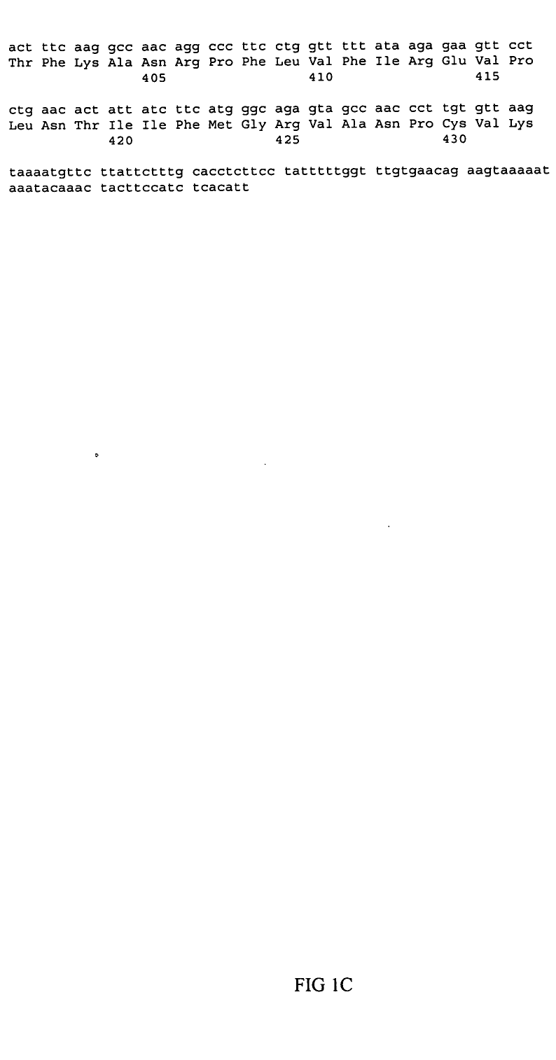

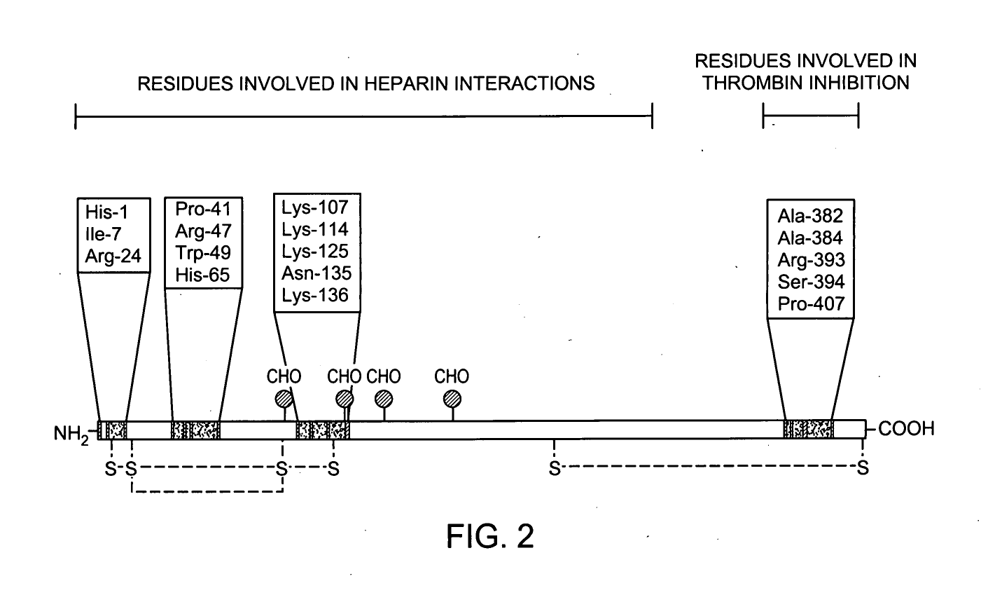

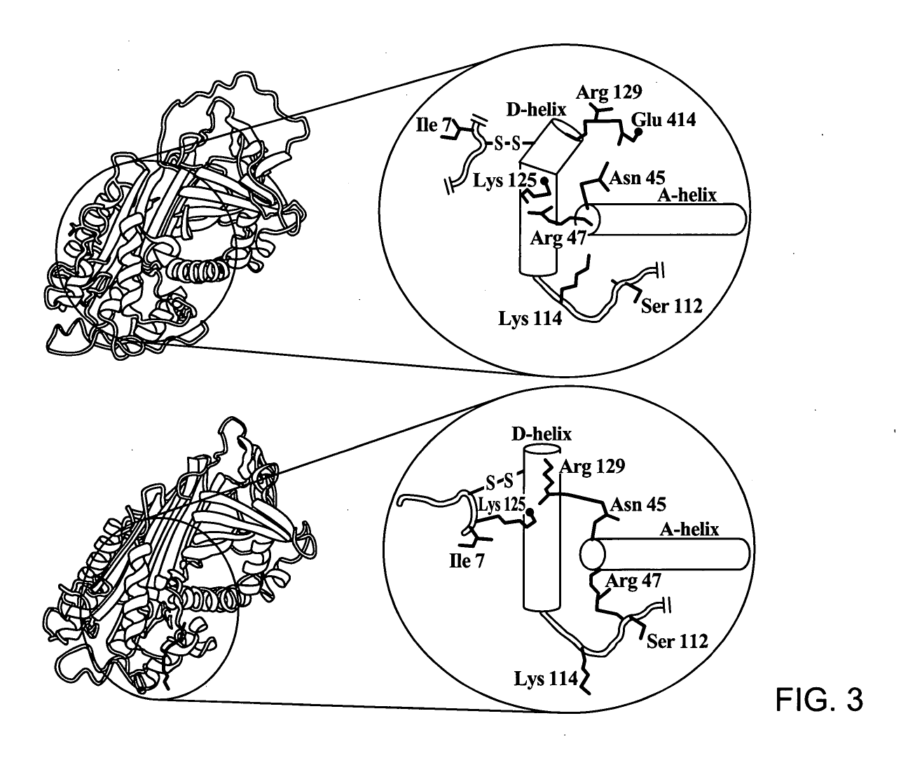

### DETAILED DESCRIPTION

1. General

The invention is based, at least in part, on the surprising finding that antithrombin III (ATIII) that has been treated to increase its molecular weight (high molecular weight ATIII) effectively reduces the viral load in HIV infected cells. Although the mechanism of action of high molecular weight ATIII is not precisely known, it is thought to act as a fusion inhibitor and/or an intracellular inhibitor or to somehow be involved in signal transduction.

2. High Molecular Weight ATIII

ATIII

The invention features pharmaceutical compositions comprised of high molecular weight ATIII and the use thereof in treating viral diseases. ATIII can be obtained, for example, from fraction IV-1 or IV, or supernatant I or II+III obtained by Cohn's fractionation of blood plasma (Lebing W R et al., Vox Sang. 67:117-24 (1994), Hoffman D L, Am. J. Med. 87: 23S-26S (1989), Wickerhauser M. et al, Vox Sang 36: 281-93 (1979) . ATIII is also commercially available (Aventis, Genzyme Transgenic Corporation Biotherapeutics, Baxter Healthcare, Calbiochem, Bayer and Sigma).

Alternatively, recombinant ATIII can be prepared, for example, using E. coli, cell culture (EP-339919), genetic engineering (EP-90505), transgenic animals (Larrik and Thomas, Curr. Opin. Biotechnol. 12:41111-8 (2001), Edmunds et al., Blood 12:4561-71 (1998), U.S. Pat. Nos. 6,441,145 and 5,843,705) and the like.

Table 1 presents nucleic acid and amino acid sequences of ATIII from various organisms, including variant nucleotide sequences, i.e. sequences that differ by one or more nucleotide substitution, addition or deletion, such as allelic variants. ATIII from mammalian species as well as variants thereof can be used to generate the high molecular weight ATIIIs of the present invention.

TABLE 1
SEQ ID NOs of Antithrombin IIIs as described herein

| | | GenRank |
|---|---|---|
| Sequence | SEQ ID NO | Accession |
| Human ATIII, nucleotide sequence | SEQ ID NO: | NM_000488 |
| Human ATIII, amino acid sequence | SEQ ID NO: | NP_000479 |
| Human ATIII variant, nucleotide | SEQ ID NO: | D29832.1 |
| sequence | | |
| Human ATIII variant, amino acid | SEQ ID NO: | BAA06212 |
| sequence | | |
| Human ATIII variant, nucleotide | SEQ ID NO: | BC022309.1 |
| sequence | | |
| Human ATIII variant, amino acid | SEQ ID NO: | AAH22309.1 |
| sequence | | |
| Human ATIII variant, nucleotide | SEQ ID NO: | AB083707 |
| sequence | | |
| Human ATIII variant, amino acid | SEQ ID NO: | BAC21176.1 |
| sequence | | |
| Ovis aries ATIII nucleotide | SEQ. ID. NO | X68287 |
| sequence | | |
| Ovis aries ATIII amino acid | SEQ. ID. NO | CAA48347.1 |
| sequence | | |
| Mus musculus ATIII nucleotide | SEQ. ID. NO | S47225 |
| sequence | | |
| Mus musculus ATIII amino acid | SEQ. ID. NO | AAB23965 |
| sequence | | |
| Sus scrofa domestica ATIII nucleotide | SEQ. ID. NO | AF281653 |
| sequence | | |
| Sus scrofa domestica ATIII amino acid | SEQ. ID. NO | JX0364 |
| sequence | | |
| Rattus norvegicus ATIII nucleotide | SEQ. ID. NO | XM_222802 |
| sequence | | |
| Rattus norvegicus ATIII amino acid | SEQ. ID. NO | XP_222802 |
| sequence | | |
| Gallus gallus ATIII nucleotide | SEQ. ID. NO | S79838 |
| sequence | | |
| Gallus gallus ATIII amino acid | SEQ. ID. NO | AAB35653.1 |
| sequence | | |
| Xenopus laevis ATIII nucleotide | SEQ. ID. NO | AF411693.1 |
| sequence | | |
| Xenopus laevis ATIII amino acid | SEQ. ID. NO | AAL60467.1 |
| sequence | | |
| Bos taurus ATIII, amino acid | SEQ ID NO: | P41361 |
| sequence | | |

One skilled in the art will appreciate that variations in one or more nucleotides (up to about 3-5% of the nucleotides) of the nucleic acids encoding a particular protein may exist among individuals of a given species due to natural allelic variation. Any and all such nucleotide variations and the encoded polypeptides can be used to prepare the super ATIIIs of the invention. For instance, it is reasonable to expect, for example, that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, scrine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W.H. Freeman and Co., 1981).

This invention further contemplates a method of generating sets of combinatorial mutants of the subject ATIII, as well as truncation mutants, and is especially useful for identifying potential variant sequences (e.g. homologs) that are functional in inhibition of viral infections. The purpose of screening such combinatorial libraries is to generate, for example, ATIII homologs with selective potency. In a representative embodiment of this method, the amino acid sequences for a population of ATIII homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential ATIII sequences. For instance, a mixture of synthetic oligonucleotides can be enzymatically ligated into gene sequences such that the degenerate set of potential ATIII nucleotide sequences are expressible as individual polypeptides, or alternatively, as a set of larger fusion proteins (e.g. for phage display). There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential ATIII sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, ATIII homologs can be generated and isolated from a library by screening using, for example, alanine scanning mutagenesis and the like (Ruf et al., (1994) Biochemistry 33:1565-1572; Wang et al., (1994) J Biol. Chem. 269:3095-3099; Balint et al., (1993) Gene 137:109-118; Grodberg et al., (1993) Eur. J Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of ATIIIs.

ATIIIs reduced to generate mimetics, e.g. peptide or non-peptide agents, which are able to mimic the anti-viral activity of modified ATIII, can also be used to generate the super ATIIIs of the invention. To illustrate, the critical residues of the subject ATIII which are involved in anti-viral activity can be determined and used to generate ATIII-derived peptidomimetics which act to inhibit viral infections. By employing, for example, scanning mutagenesis to map the amino acid residues of the subject ATIII which are involved in viral inhibition, peptidomimetic compounds can be generated which mimic those residues involved in viral inhibition. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J Med. Chem. 29:295; and Ewenson et al., in Peptides: Structure and Function (Proceedings of the 9th American Peptide Symposium) Pierce Chemical Co. Rockland, IL, 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

ATIII may be substantially purified by a variety of methods that are well known to those skilled in the art. Substantially pure protein may be obtained by following known procedures for protein purification, wherein an immunological, chromatographic, enzymatic or other assay is used to monitor purification at each stage in the procedure. Protein purification methods are well known in the art, and are described, for example in Deutscher et al., Guide to Protein Purification, Harcourt Brace Jovanovich, San Diego (1990). ATIII can also be purified by a method described in, for example, U.S. Pat. No. 3,842,061 and U.S. Pat. No. 4,340,589.

As used herein, the term “substantially purified,” refers to ATIII that has been separated from components which naturally accompany it. Preferably the ATIII is at least about 80%, more preferably at least about 90%, and most preferably at least about 99% of the total material (by volume, by wet or dry weight, or by mole percent or mole fraction) in a sample. Purity can be measured by any appropriate method, e.g., in the case of polypeptides by column chromatography, gel electrophoresis or HPLC analysis.

High Molecular Weight ATIII

As shown herein, administration to a subject of ATIII that has been treated in a manner that results in an increased molecular weight reduces the viral load in viral infected cells. Particularly preferred high molecular weight ATIII molecules reduce the viral load by at least 1.5 log, more preferably at least 2, 3, 4 or 5 logs better than native ATIII.

Preferred high molecular weight ATIII molecules or molecular combinations weigh in the range of about 60 kD to about 550 kD (native ATIII is 58 kD). Particularly preferred high molecular weight ATIIIs weigh in the range of at least about 60-70, 70-80, 80-90, 90-100, 100-110, 110-120, 130-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, 490-500, 500-510, 510-520, 520-530, 530-540 or 540-550.

A high molecular weight ATIII can be prepared, for example, as shown in Example 1, by heat treatment and association with an oligosugar, such as heparin. Heat treatment may include heating at 60° C. or more for at least about 30 minutes, more preferably for a number of hours. Preparation of heat treated ATIII is described in Larsson et al., J Biol. Chem. 276:11996-12002 (2001).

“Oligosugar” as used herein refers to monosaccharides, disaccharides, and polysaccharides (including penta-, hepta- and hexa-saccharides), sugar alcohols, and amino sugars. Examples of monosaccharides include glucose, fructose, galactose, mannose, arabinose, and inositol. Examples of disaccharides include saccharose, lactose, maltose, pectin. Examples of sugar alcohols include mannitol, sorbitol, and xylitol. Examples of amino sugars include glucosamine, galactosamine, N-acetyl-D-glucosamine and N-acetyl galactosamine, which are the building blocks that can form more complex oligosugars, such as aminoglycosides and heparin. Preferred oligosugars are heparin (low molecular weight (2-4 kDa) and high molecular weight (at least 12 kDa), pectin, pentasaccharides and aminoglycosides. Preferred oligosugars have an affinity for ATIII. For example, heparin is known to interact with ATIII at certain sites, including His-1, Ile-7, Arg 24, Pro-41, Asn-45, Arg-47, Trp-49, His-65, Lys-107, Ser-112, Lys-114, Phe-121, Phe-122, Lys-125, Arg-129, Asn-135, Lys -136, Glu 414 amino acids of ATIII (Pratt et al., Seminars in Hematology. 28:3-9 (1991), Skinner et al., J Mol. Biol. 266:601-609 (1997), Jairajpuri et al., J. Biol. Chem. M212319200 (2003)). Oligosugars as used herein can be derivatized with additional small molecules, such as biotin, avidin or streptavidin.

Oligosugars may be linked to a heat treated ATIII molecule through incubation at 37-60° C. for 1-72 hours in buffers such as 0.02 M sodium phosphate, 0.05 M NaCl, pH 7.4. Likewise, the association of ATIII with oligosugars such as heparin may be accomplished using standard synthetic organic chemistry methods well known to those skilled in the art (See for example, March J. Advanced Organic Chemistry, John Wiley & Sons , Inc. (1992)). High molecular weight ATIII can be generated by heat modifying ATIII and then adding an oligosugar or first treating the ATIII with an oligosugar and then heat modifying as shown in the examples.

High molecular weight ATIIIs can also be prepared by conjugating an ATIII molecule with at least one other ATIII molecule, to create a multimer, such as a dimer or a trimer. In addition, functional fragments of ATIII may be conjugated to other functional fragments or to full length molecules to generate high molecular weight ATIII.

Additional high molecular weight ATIIIs can be prepared based on association with sulfated molecules (Gunnarsson, G T and U R Desai, Bioorg Me Chem Lett 13(4): 679-893 (2003)).

High molecular weight ATIII can be formulated in a manner that extends its in vivo half life. For example, high molecular weight ATIII can be attached to an additional large molecular weight molecule, such as a protein or polymer that allows longer blood circulation and slower release. Preferably the controlled release formulation is comprised of an amid or polymeric product that is biodegradable.

Controlled release formulations may include implants and microencapsulated delivery systems (see WO 94/23697 and U.S. Pat. No. 5,102,872 respectively). High molecular weight ATIII may be entrapped or conjugated to polymers and implanted in a patient to facilitate slow release. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Examples of these technologies are shown in U.S. Pat. Nos. 5,110,596, 5,034,229, and 5,057,318, the respective contents of which are hereby incorporated by reference.

Other controlled release formulations may include transdermal delivery systems. Examples of these may include the microsealed system which is a partition-controlled delivery system that contains a reservoir with a saturated suspension of modified ATIII in a water-miscible solvent homogeneously dispersed in a silicone elastomer matrix. A second system is the matrix-diffusion controlled system. The third and most widely used system for transdermal drug delivery is the membrane-permeation controlled system. A fourth system, recently made available, is the gradient-charged system. Additionally, advanced transdermal carriers include systems such as iontophoretic and sonophoretic systems, thermosetting gels, and prodrugs (see Ranade VV. (1991) J. Clin Pharmacol 31(5):401-418) In these system, absorption promoters may be used to enhance the penetration of modified ATIII through the skin.

The absorption promoters may be selected in particular, from propylene glycol, hexylene glycol, propylene glycol dipelargonate, glyceryl monoethyl ether, diethylene glycol, monoglycerides, monooleate of ethoxylated glycerides (with 8 to 10 ethylene oxide units), Azone (1-dodecylazacycloheptan-2-one), 2-(n-nonyl)-1,3-dioxolane, isopropylmyristate, octylmyristate, dodecyl-myristate, myristyl alcohol, lauryl alcohol, lauric acid, lauryl lactate, terpinol, 1-menthol, d-limonene, .beta.-cyclodextrin and its derivatives or surfactants such as polysorbates, sorbitan esters, sucrose esters, fatty acids, bile salts, or alternatively lipophilic and/or hydrophilic and/or amphiphilic products such as poly-glycerol esters, N-methylpyrrolidone, polyglycosylated glycerides and cetyl lactate. The absorption promoter preferably represents from 5 to 25% of the weight of the composition. Further description of absorption promoters appears in in U.S. Pat. No. 6,538,039.

Activity Assays

High molecular weight ATIII as described herein, may be assayed for antiviral activity using any of a number of commercially available assays. For example, the ability to reduce HIV viral load may be determined using the Alliance® HIV-1 p24 enzyme linked immunosorbent assay, for example as shown in Example 2. In other embodiments, one skilled in the art could determine HIV-1 inhibitory activities of modified ATIII by detecting the presence and/or relative amount of viral DNA using for example, RT-PCR (Amplicor HIV-1 Monitor; Roche Diagnostic Systems), nucleic acid sequence based amplification (HIV-1 RNA QT; Organon Teknika), nucleic acid hybridization and branched DNA signal amplification (Quantiplex HIV-1 RNA; Bayer Nucleic Acid Diagnostics), DNA hybridization and colorimetric detection (Digene assay: Digene Diagnostics), a multiplex transcription-mediated amplification system (Gen-Probe), and nucleic acid and sequence based amplification assays (Nuclisens).

Inhibition of Hepatitis A by modified ATIII may be identified, for example, using commercially available radioimmunoassay (RIA) or ELISA assays to detect specific Hepatitis A Virus antibody of the IgM class, molecular hybridization and PCR detection techniques. Inhibition of Hepatitis B may be assayed, for example, by liquid hybridization tests (Genostics Assay; Abbott Laboratories, Chicago, Ill.), branched DNA assays (Bayer, Emeryville, Calif.), and PCR assays (Cobas Amplicor HBV Monitor or Cobas-AM). Inhibition of Hepatitis C may be assayed using ELISA assays specific for Hepatitis C virus, RNA detection by standardized RT-PCR assays (Amplicor HCV 2.0; Roche Molecular Systems), and branched DNA assays (Quantiplex HCV RNA 2.0; Chiron Diagnostic Laboratories) (Clinical Virology, 2^{nd} Ed., by Richman, Whitley, Hayden (American Society for Microbiology Press: 2002 Chapters 30, 32, 46, 52). Inhibition of coronaviruses (e.g. SARS) may be assayed using commercially available PCR assays.

3. Pharmaceutical Compositions and Therapeutic Uses

Based on the bioactivity shown herein, pharmaceutical preparations comprising an effective amount of high molecular weight ATIII can be administered to subjects (including humans and animals, such as cows, horses, dogs, cats, etc.) to treat the subject for an infection and/or inflammation. Preferably the infection is bacterial or viral based. Particularly preferred viral infections are retroviral infections, caused, for example, by viruses selected from the group consisting of: HIV, HAV, HBV, HCV, and SARS.

In addition to the utility as an anti-infective/anti-inflammatory agent, the pharmaceutical compositions of the present invention may also be used to inhibit thrombin activation in a patient in need of such inhibition. Thrombin activation related diseases in a -patient include sepsis, trauma, acute respiratory distress syndrome, thrombosis, stroke, and restenosis. The pharmaceutical compositions may also be used to treat patients at risk of a thrombin related pathological disease such as reocclusion and restenosis in percutaneous transluminal coronary angioplasty; thrombosis associated with surgery, ischemia/reperfusion injury; and coagulation abnormalities in cancer or surgical patients. The pharmaceutical compositions may further be used as anti-coagulants in the treatment of for example, congenital antithrombin III deficiency which leads to an increased risk of venous and arterial thrombosis, or acquired antithrombin III deficiency which results in disseminated intravascular coagulation, microangiopathic hemolytic anemias due to endothelial damage (i.e. hemolytic-uremic syndrome) and veno-occlusive disease (VOD).

Pharmaceutical compositions of high molecular weight ATIII may be formulated in a conventional manner using one or more physiologically acceptable carriers or excipients. Thus, high molecular weight ATIII may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The high molecular weight ATIII may be formulated for parenteral administration by injection, e.g., by bolus injection or continuous infusion. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The high molecular weight ATIII may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possiblity of local noninvasive delivery of drugs over an extended period of time. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories.

The mixture of high molecular weight ATIII and pharmacologically acceptable additives is preferably prepared as a lyophilized product, and dissolved when in use. Such preparation can be prepared into a solution containing about 1-100 units/ml of high molecular weight ATIII, by dissolving it in distilled water for injection or sterile purified water. More preferably, it is adjusted to have a physiologically isotonic salt concentration and a physiologically desirable pH value (pH 6-8).

ATIII has been shown to be well-tolerated when administered at a dose of ˜100 U/kg/day (Warren et al., JAMA 286: 1869-78 (2001)) and has an overall elimination half-life with 18.6 h was demonstrated (Ilias et al. Intensive Care Medicine 26: 7104-7115 (2000)). While the dose is appropriately determined depending on symptom, body weight, sex, animal species and the like, it is generally 1-1,000 units/kg body weight/day, preferably 10-500 units/kg body weight/day of ATIII for a human adult, which is administered in one to several doses a day. In the case of intravenous administration, for example, the dose is preferably 10-100 units/kg body weight/day.

Furthermore, as those skilled in the art will understand, the dosage of any agent, compound, drug, etc., of the present invention will vary depending on the symptoms, age and body weight of the patient, the nature and severity of the disorder to be treated or prevented, the route of administration, and the form of the supplement. Any of the subject formulations may be administered in any suitable dose, such as, for example, in a single dose or in divided doses. Dosages for the compounds of the present invention, alone or together with any other compound of the present invention, or in combination with any compound deemed useful for the particular disorder, disease or condition sought to be treated, may be readily determined by techniques known to those of skill in the art, based on the present description, and as taught herein. Also, the present invention provides mixtures of more than one subject compound, as well as other therapeutic agents.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The high molecular weight ATIII of the present invention, may also be formulated in combination with other anti-viral drugs. For example, the high molecular weight ATIII can be formulated with reverse transcriptase inhibitors, including cocktails, such as highly active antiretroviral drug therapy (HAART) regimen (zidovudine, zalcitabine, didanosine, stavudine, lamivudine, abacavir, tenofovir, nevirapine, efavirenz, delavirdine) and protease inhibitors (saquinavir, ritonavir, indinavir, nelfinavir, amprenavir, lopinavir), adenine arabinoside, adenine arabinoside 5′-monophosphate, acyclovir, ganciclovir, famciclovir, lamivudine, clevudine, afedovir dipivoxil, entecavir, IFN-α-2b, IFN-α-2a, lymphoblastoid IFN, consensus-IFN, IFN-β, IFN-γ, pegylated IFN-α-2a, corticosteroids, or thymosin al, IL-2, IL-12, ribavirin, cyclosporin or granulocyte macrophage colony stimulating factor.

The combined use of pharmaceutical compounds of the present invention and other antivirals may reduce the required dosage for any individual component because the onset and duration of effect of the different components may be complimentary. In such combined therapy, the different active agents may be delivered together or separately, and simultaneously or at different times within the day.

Exemplification

The invention now being generally described, it will be more readily understood by reference to the following examples, which are included merely for purposes of illustration of certain aspects and embodiments of the present invention, and are not intended to limit the invention.

### EXAMPLE 1

### Preparation of High Molecular Weight ATIII

The following examples describe methods used to prepare certain high molecular weight ATIII.

1. Preparation of Heat Treated ATIII (Form 1)

10 mg of ATIII was dissolved (or diluted) in 2 ml of 10 mM Tris/HCI, 0.5 M sodium citrate, pH 7.4 and incubated for 24 h at 60° C. with very gentle stirring. The incubate was dialyzed with a use 15 KDa membrane size dialyse membrane against 0.02 M sodium phosphate, 0.05 M NaCl, pH 7.4. The dialyzed protein was used in the inhibition tests or incubated with heparin (see below) to produce high molecular weight form 2.

2. Oligosugar Activation of Heat Treated-ATIII (Form 2 )

ATIII (Form 1) was incubated with a 1:1 mixture (w/w) of Low Molecular Weight (MW) Heparin (Sigma) at 370° C. for 24-48 hours in 0.02 M sodium phosphate, 0.05 M NaCl, pH 7.4. Afterwards the solution was dialyzed against 0.02 M sodium phosphate, 0.05 M NaCl, pH 7.4. The dialyzed protein was used in the below described inhibition test to determine antiviral activity.

3. Oligosugar Activation of ATIII (Form 3)

ATIII was incubated with a 1:1 mixture (w/w) of Low MW Heparin (Sigma) at 370° C. for 24-48 hours in 0.02 M sodium phosphate, 0.05 M NaCl, pH 7.4. Afterwards the solution was dialyzed against 0.02 M sodium phosphate, 0.05 M NaCI, pH 7.4. The dialyzed protein was used in the below described inhibition test to determine antiviral activity.

4. Heat Treatment of Oligosugar Activated-ATIII (Form 4)

Form 3 was dialyzed in 10 mM Tris/HCI, 0.5 M sodium citrate, pH 7.4 and incubated for 24 h at 60° C. with very gentle stirring. Afterwards the incubate was dialyzed using a 30-50 kD or larger membrane against 0.02 M sodium phosphate, 0.05 M NaCl, pH 7.4. The dialyzed protein was used in the below described inhibition test to determine antiviral activity.

### EXAMPLE 2

### Evaluation of HIV-1 Inhibitory Activity

X4 HTLV-IIIB (hereinafter X4 HIV; Chang et al., NATURE, 363: 466-9 (1993)), a prototypical T-tropic strain of HIV (American Type Tissue Collection, Monassass, Va., USA; ATCC No. CRL-8543), was used to assess the effect of wildtype and high molecular weight ATIII on T-tropic HIV infection. The quantity of virus in a specified suspension volume (eg. 0.1 ml) that will infect 50% of a number (n) of cell culture microplate wells, or tubes, is termed the Tissue Culture Infectious Dose 50 [TCID₅₀]. TCID₅₀ is used as an alternative to determining virus titer by plaqueing (which gives values as PFUs or plaque-forming units). Human T lymphoblastoid cells (H9 cells) expressing the human leukocyte antigen proteins (HLA) B6, Bw62, and Cw3 were acutely infected with X4 HIV at a MOI of 1×10⁻² TCID₅₀ per milliliter. The infected H9 cells were resuspended to 5×10⁵ cells/ml in R20 cell culture medium. Two milliliters of this suspension was pipetted into each well of a 24-well microtiter plate. These cells were then cultured in the presence or absence of various forms of wildtype and high molecular weight ATIII for up to 12 days. Every three days (days 3, 6, 9 and 12), 1 ml cell supernatant was removed from test wells and replaced with an equal volume of R20 cell culture medium. Control wells were similarly sampled but received media containing untreated ATIII.

The concentration of the viral core protein p24 (gag) for HIV (Alliance® HIV-1 p24 ELISA kit, NEN® Life Science, Boston, Mass., USA) was measured for each sample obtained at days 0, 3, 6, 9 and 12 respectively.

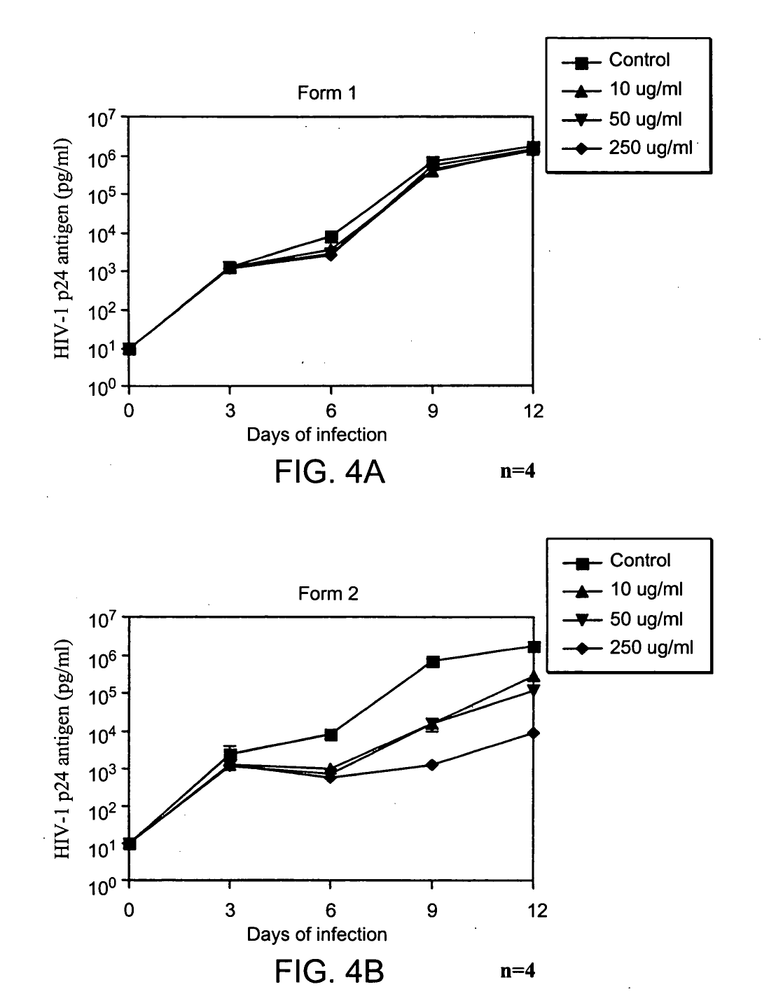

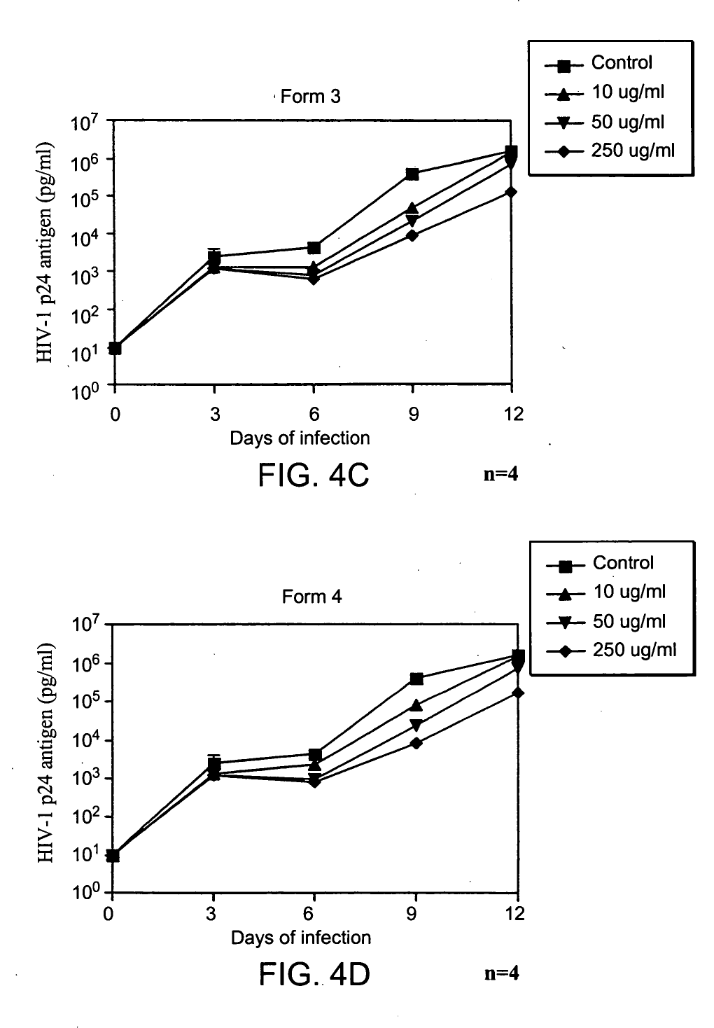

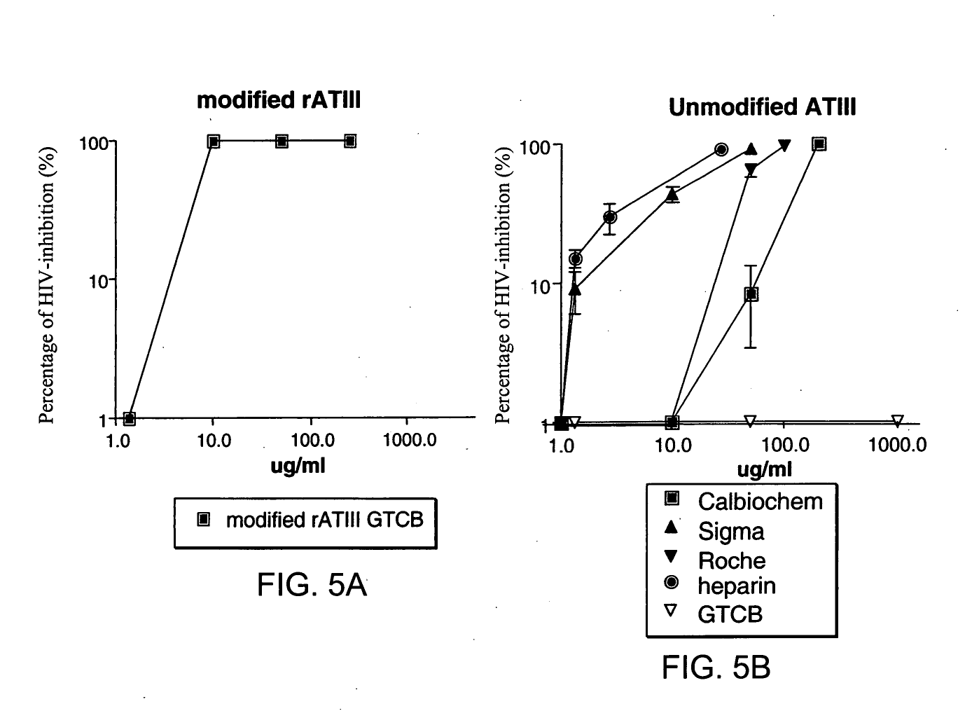

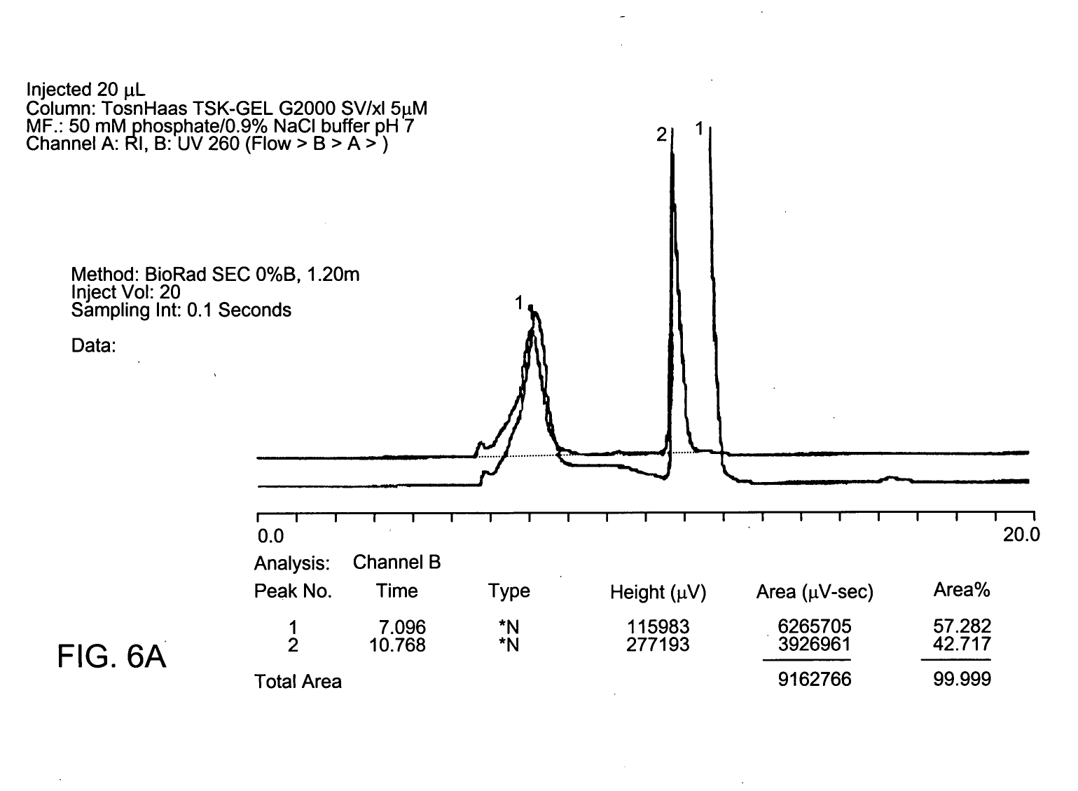

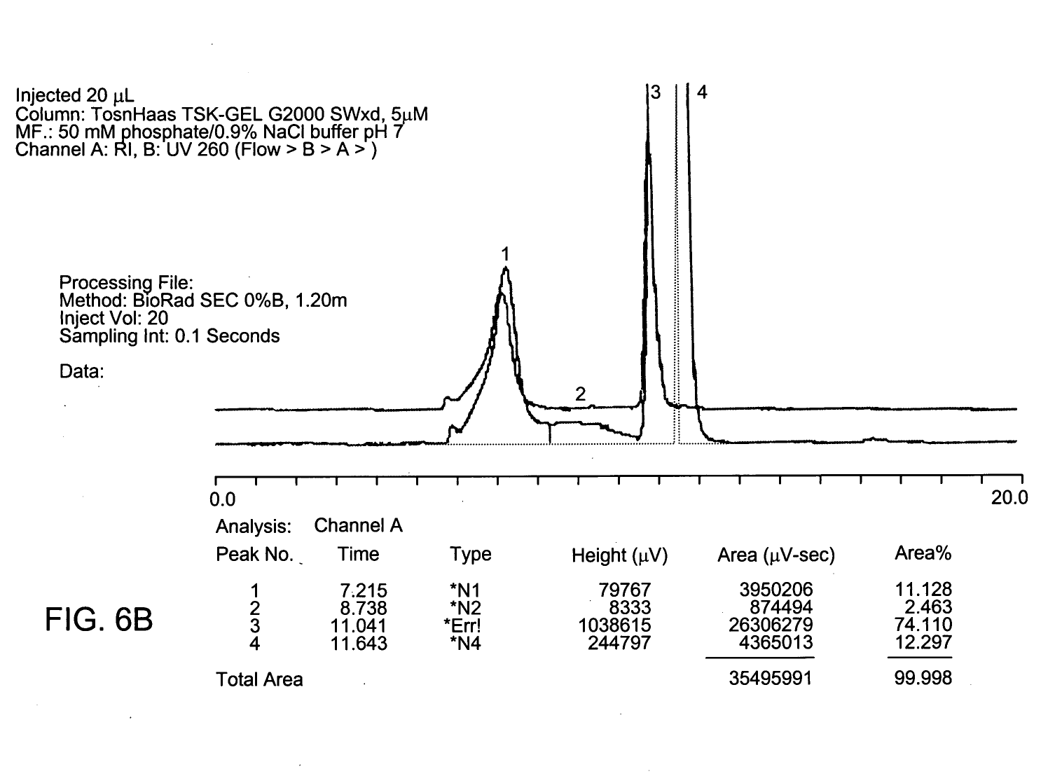

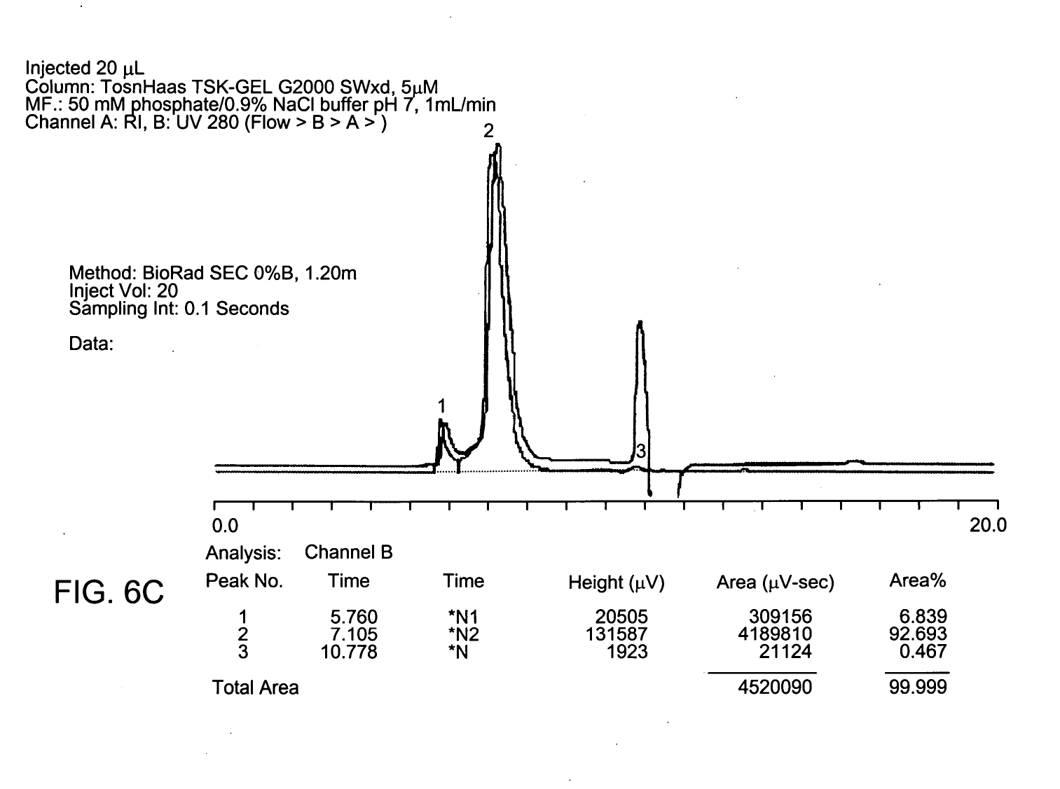

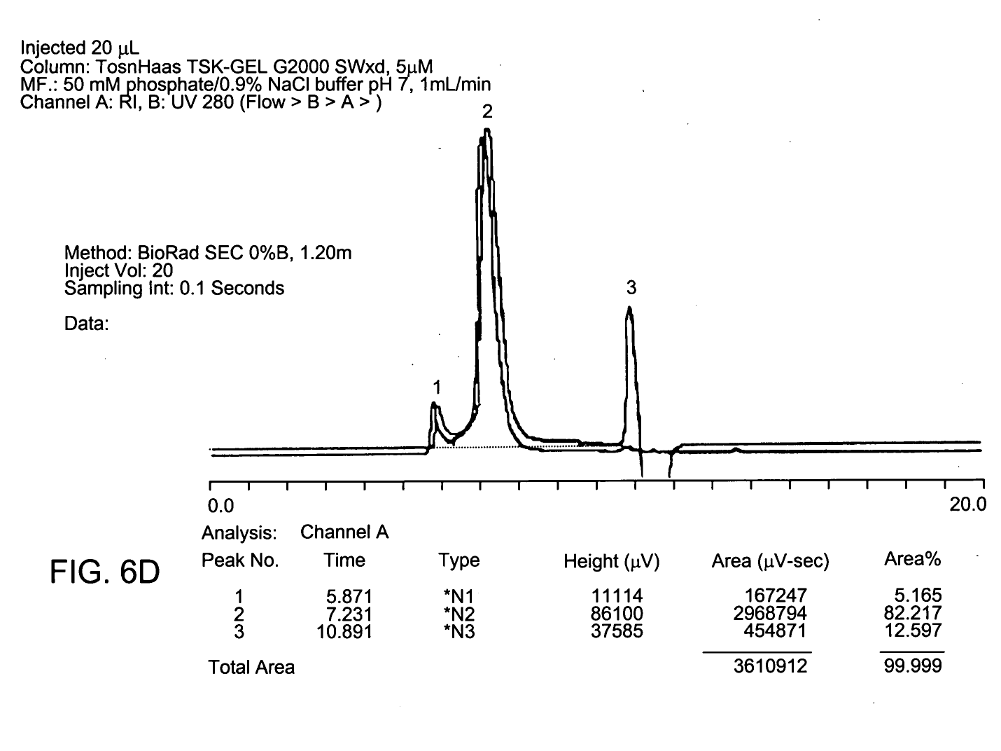

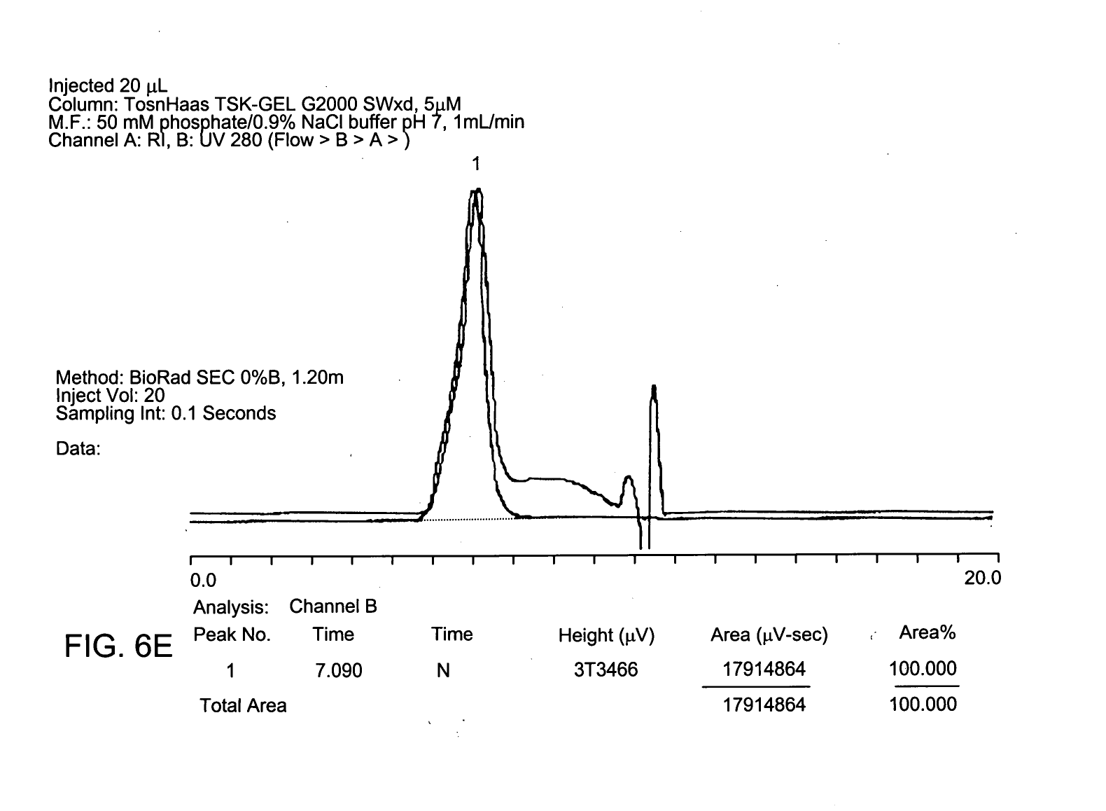

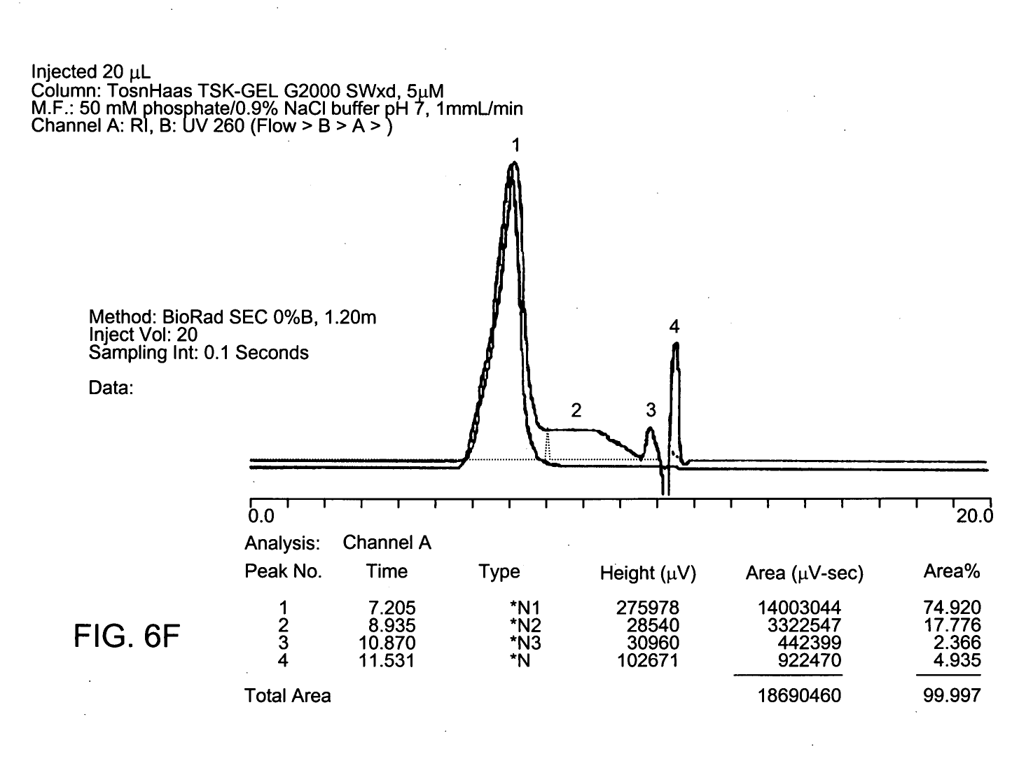

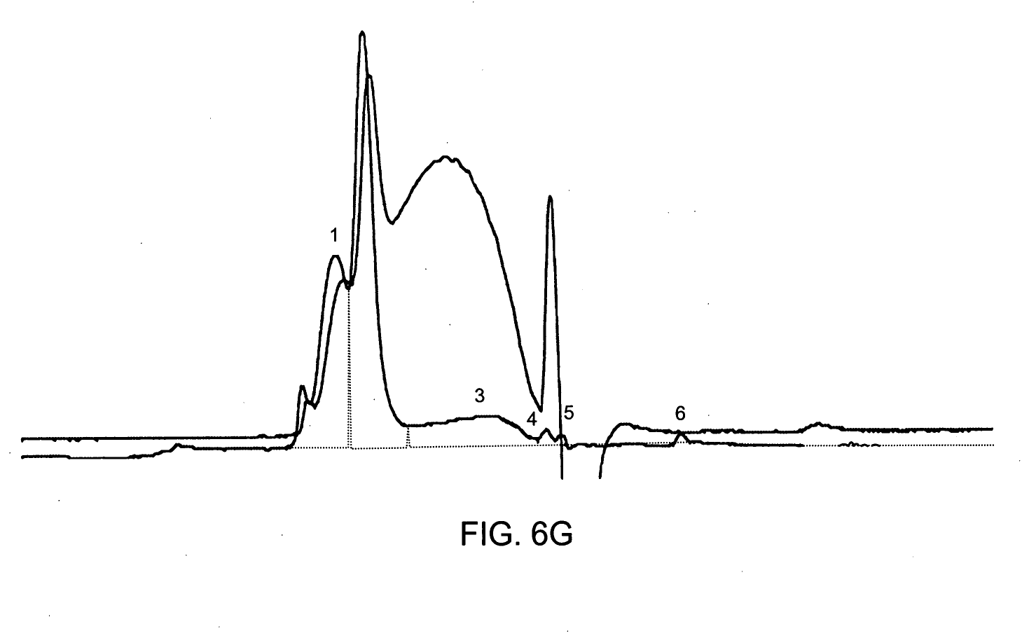

The results which are shown in FIGS. 4 and 5 demonstrate that the various forms of high molecular weight ATIII have the most potent HIV-1 inhibitory activity, whereas unmodified ATIII from GTC Biotherapeutics and Aventis showed virtually no anti-viral activity.

### EXAMPLE 3

### HPLC Analysis of High Molecular Weight ATIII

| | |
|---|---|
| HPLC system: | Varian ProStar 210 Pumps, Milton Roy |
| | Spectromonitor 3000 UVDetector, BioRad 1755 |
| | Refractive Index Monitor. |
| Column: | TSK-gel G2000-SWx1 (30 mm × ill 7.8 mm, 5˜) |
| Elution: | 0.05 M phosphate pH 7.0, 0.9% NaCl, 1 ml/min |
| Detection: | Channel A - RI detector |
| | Channel B - UV detector (280 nm) |
| MW calibration: | BioRad protein standards. |

| | Molecular | |
|---|---|---|
| Protein | Weight (MW)* | Retention Time (RT) |
| Thyroglobulin | (669,000) | 5.708 |
| IgG | (160,000) | 6.521 |
| Ovalbumin | (44,300) | 7.521 |
| Myoglobin | (17,600) | 9.228 |
| Vitamin B12 | (1,355) | 11.385 |
| | Relative MW* | |
| Sample description: | (main peaks (%)) | UV RT |
| #1 - ATIII control | 68,100 | 7.098 (90) |
| #2 - ATIII heat-treated | 74,500 | 7.016(51); 6,431(32); |
| | | 9.431(16) |
| #3 - ATIII heat-treated | 74,500 | 7.008 + 6.616(80); |
| followed by heparin | | 9,485(13), 5.8 (7) |
| incubation | | |
| #4 - ATIII heparin incubation | 67,600 | 7,105(93); 5,760(7) |
| #5 - ATIII heparin incubation | 68,600 | 7,090 + 5,800 (100) |
| followed by heat | | |
| treatment | | |
| #6 - Protein calibration | | |
| standards (see above) | | |

*The relative molecular weight (MW) is calculated based on the calibration plot obtained for BioRad protein standards

### SUMMARY

The present results show that the degree of modification of ATIII increases in a row in the following manner #3>#2>#5>#4 that correlates with THE accumulation of heparin in the protein containing polymer fraction (RT>7.8) and increase in RT of major protein fraction (RT˜7.0˜7.1, UV at 280 nm). The relative change in MW of major fraction of ATIII conjugate compared to starting ATIII is reported in the table above. (The MW of glycosylated ATIII is ˜54,000 Da. MW of not glycosilated ATIII (alpha and beta isoforms correspondingly 47,800 Da and 46,800 Da).

All ATIII modifications were found to contain substantial amounts of high molecular weight protein aggregates, which are in exclusion volume for TSK G2000 column, but might be analyzed on columns with better resolution for high MW polymers.

The practice of the present invention will employ, unless otherwise indicated, conventional techniques of virology, protein chemistry, cell biology, cell culture, molecular biology, microbiology, and recombinant DNA, which are within the skill of the art. Such techniques are explained fully in the literature. See, for example, Clinical Virology, 2^{nd} Ed., by Richman, Whitley, Hayden (American Society for Microbiology Press: 2002), Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B.Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); and Methods In Enzymology, Vols. 154 and 155 (Wu et al. eds.)All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A pharmaceutical composition comprising: a pharmaceutically acceptable carrier and an effective amount of antithrombin III (ATIII) having a molecular weight in the range of 60-550 kD and an ability to reduce the load in virally infected cells.

2. The pharmaceutical composition of claim 1 wherein the ATIII has been heat treated and modified by an oligosugar.

3. The pharmaceutical composition of claim 2 wherein the heat treatment is at least 60° C. or more for at least 30 minutes.

4. The pharmaceutical composition of claim 2 wherein the oligosugar is a monosaccharide.

5. The pharmaceutical composition of claim 2 wherein the oligosugar is a polysaccharide.

6. The pharmaceutical composition of claim 2 wherein the oligosugar is a low molecular weight heparin

7. The pharmaceutical composition of claim 2 wherein the oligosugar is a high molecular weight heparin.

8. The pharmaceutical composition of claim 2 wherein the oligosugar is pectin.

9. The pharmaceutical composition of claim 2 wherein the oligosugar is an amino glycoside.

10. The pharmaceutical composition of claim 2 wherein the oligosugar is derivatized with biotin.

11. The pharmaceutical composition of claim 1, which is an ATIII multimer.

12. The pharmaceutical composition of claim 1, which is modified by a sulfated molecule.

13. The pharmaceutical composition of claim 1, wherein the retroviral infection is a Hepatitis A Virus (HAV) infection.

14. The pharmaceutical composition of claim 1, wherein the retroviral infection is a Hepatitis B Virus (HBV) infection.

15. The pharmaceutical composition of claim 1, wherein the retroviral infection is a Hepatitis C Virus (HCV) infection.

16. The pharmaceutical composition of claim 1, wherein the retroviral infection is a Human Immunodeficiency Virus (HIV) infection.

17. The pharmaceutical composition of claim 1, wherein the retroviral infection is a corona virus infection.

18. The pharmaceutical composition of claim 1 wherein the high molecular weight ATIII is a dimer.

19. The pharmaceutical composition of claim 1, which is in a controlled release formulation.

20. The pharmaceutical composition of claim 19, wherein the controlled release formulation includes a biodegradable polymer.

21. A method of treating HIV infection in a subject, comprising the step of: administering to the subject a therapeutically effective amount of a pharmaceutical composition of claim 1.

22. A method of claim 19, wherein the pharmaceutical composition is in the range of 10-250 mgs per unit dose.

23. The method of claim 22, wherein the pharmaceutical composition is administered to the patient 16 to 17 times per day.

24. The method of claim 21, wherein the administration occurs once a week.

25. The method of claim 22, wherein the administration occurs at least two times per week.

26. The method of claim 21 wherein the pharmaceutical composition of claim 1 is used in combination with another anti-viral drug.

27. The method of claim 26 wherein the other anti-viral drug is a Highly Active Antiretroviral Drug Therapy (HAART) agent.

28. A method of treating Hepatitis A viral infection in a subject, comprising the step of: administering to a subject with Hepatitis A viral infection a therapeutically effective amount of a pharmaceutical composition of claim 1.

29. The method of claim 28 wherein the pharmaceutical composition of claim 1 is used in combination with an anti-viral drug.

30. A method of treating Hepatitis B viral infection in a subject, comprising the step of: administering to a subject with Hepatitis B viral infection a therapeutically effective amount of a pharmaceutical composition of claim 1.

31. The method of claim 30 wherein the pharmaceutical composition of claim 1 is used in combination with an anti-viral drug.

32. The method of claim 31 wherein the pharmaceutical composition of claim 1 is used in combination with an interferon or interferon derived drug.

33. A method of treating Hepatitis C viral infection in a subject, comprising the step of: administering to a subject with Hepatitis C viral infection a therapeutically effective amount of a pharmaceutical composition of claim 1.

34. The method of claim 33 wherein the pharmaceutical composition of claim 1 is used in combination with an anti-viral drug.

35. The method of claim 33 wherein the pharmaceutical composition of claim 1 is used in combination with an interferon or interferon derived drug.

36. A method of treating HIV-1 infection in a subject, comprising the step of: administering to the subject a therapeutically effective amount of a pharmaceutical composition of claim 16.

37. The method of claim 36 wherein the pharmaceutical composition of claim 16 is used in combination with an anti-viral drug.

38. The method of claim 37 wherein the anti-viral drug is a Highly Active Antiretroviral Drug Therapy (HAART) agent.

39. A method of treating Hepatitis A viral infection in a subject, comprising the step of: administering to the subject a therapeutically effective amount of a pharmaceutical composition of claim 16.

40. The method of claim 39 wherein the pharmaceutical composition of claim 16 is used in combination with an anti-viral drug.

41. A method of treating Hepatitis B viral infection in a subject, comprising the step of: administering to the subject a therapeutically effective amount of a pharmaceutical composition of claim 16.

42. The method of claim 41, wherein the pharmaceutical composition of claim 16 is used in combination with an anti-viral drug.

43. The method of claim 42 wherein the anti-viral drug is an interferon or interferon derived drug.

44. A method of treating Hepatitis C viral infection in a subject, comprising the step of: administering to a subject with Hepatitis C viral infection a therapeutically effective amount of a pharmaceutical composition of claim 16.

45. The method of claim 44 wherein the pharmaceutical composition of claim 16 is used in combination with an anti-viral drug.

46. The method of claim 45, wherein the anti-viral drug is an interferon or interferon derived drug.

47. A kit, comprising in one or more containers and the pharmaceutical composition of claim 1.

48. A kit, comprising in one or more containers and the pharmaceutical composition of claim 16.

49. A pharmaceutical composition comprising: a pharmaceutically acceptable carrier and ATIII having a molecular weight in the range of 60-550 kD in an effective amount to treat a subject with a disease or condition that is caused by or contributed to by thrombin activation.

50. A pharmaceutical composition of claim 49, wherein the disease or condition is selected from the group consisting of: sepsis, trauma, acute respiratory distress syndrome, thrombosis, stroke, restenosis, reocclusion and restenosis in percutaneous transluminal coronary angioplasty; thrombosis associated with surgery, ischemia/reperfusion injury; and coagulation abnormalities in cancer or surgical patients, an antithrombin III deficiency, venous or arterial thrombosis, disseminated intravascular coagulation, microangiopathic hemolytic anemias and veno-occlusive disease (VOD).

51. A method of treating a disease or condition which is caused by or contributed to by thrombin activation in a subject comprising administering to the subject a pharmaceutical composition of claim 49.


SEQUENCE LISTING (22 sequences)

SEQ ID NO: 1 - DNA, 1599 bp, Homo sapiens
  CDS (121)..(1512)
  sig_peptide (121)..(216)
  mat_peptide (217)..(1512)
caccagcatcatctcctccaattcatccagctactctgcccatgaagataatagttttcaggcggattgcctcagatcacactatctccacttgcccagccctgtggaagattagcggccatgtattccaatgtgataggaactgtaacctctggaaaaaggaaggtttatcttttgtccttgctgctcattggcttctgggactgcgtgacctgtcacgggagccctgtggacatctgcacagccaagccgcgggacattcccatgaatcccatgtgcatttaccgctccccggagaagaaggcaactgaggatgagggctcagaacagaagatcccggaggccaccaaccggcgtgtctgggaactgtccaaggccaattcccgctttgctaccactttctatcagcacctggcagattccaagaatgacaatgataacattttcctgtcacccctgagtatctccacggcttttgctatgaccaagctgggtgcctgtaatgacaccctccagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgagacctaccaggacatcagtgagttggtatatggagccaagctccagcccctggacttcaaggaaaatgcagagcaatccagagcggccatcaacaaatgggtgtccaataagaccgaaggccgaatcaccgatgtcattccctcggaagccatcaatgagctcactgttctggtgctggttaacaccatttacttcaagggcctgtggaagtcaaagttcagccctgagaacacaaggaaggaactgttctacaaggctgatggagagtcgtgttcagcatctatgatgtaccaggaaggcaagttccgttatcggcgcgtggctgaaggcacccaggtgcttgagttgcccttcaaaggtgatgacatcaccatggtcctcatcttgcccaagcctgagaagagcctggccaaggtggagaaggaactcaccccagaggtgctgcaggagtggctggatgaattggaggagatgatgctggtggttcacatgccccgcttccgcattgaggacggcttcagtttgaaggagcagctgcaagacatgggccttgtcgatctgttcagccctgaaaagtccaaactcccaggtattgttgcagaaggccgagatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacaggcccttcctggtttttataagagaagttcctctgaacactattatcttcatgggcagagtagccaacccttgtgttaagtaaaatgttcttattctttgcacctcttcctatttttggtttgtgaacagaagtaaaaataaatacaaactacttccatctcacatt

SEQ ID NO: 2 - protein, 464 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNETYQDISELVYGAKLQPLDFKENAEQSRAAINKWVSNKTEGRITDVIPSEAINELTVLVLVNTIYFKGLWKSKFSPENTRKELFYKADGESCSASMMYQEGKFRYRRVAEGTQVLELPFKGDDITMVLILPKPEKSLAKVEKELTPEVLQEWLDELEEMMLVVHMPRFRIEDGFSLKEQLQDMGLVDLFSPEKSKLPGIVAEGRDDLYVSDAFHKAFLEVNEEGSEAAASTAVVIAGRSLNPNRVTFKANRPFLVFIREVPLNTIIFMGRVANPCVK

SEQ ID NO: 3 - DNA, 1467 bp, Homo sapiens
gaattcgagctcgccccggccatgtattccaatgtgataggaactgtaacctctggaaaaaggaaggtttatctcttgtccttgctgctcattggcttctgggactgcgtgacctgtcacgggagccctgtggacatctgcacagccaagccgcgggacattcccatgaatcccatgtgcatttaccgctccccggagaagaaggcaactgaggatgagggctcagaacagaagatcccggaggccaccaacaaccggcgtgtctgggaactgtccaaggccaattcccgctttgctaccactttctatcagcacctggcagattccaagaatgacaatgataacattttcctgtcacccctgagtatctctacggcttttgctatgaccaagctgggtgcctgtaatgacaccctccagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgagacctaccaggacatcagtgagttggtatatggagccaagctccagcccctggacttcaaggaaaatgcagagcaatccagagcggccatcaacaaatgggtgtccaataagaccgaaggccgaatcaccgatgtcattccctcggaagccatcaatgagctcactgttctggtgctggttaacaccatttacttcaagggcctgtggaagtcaaagttcagccctgagaacacaaggaaggaactgttctacaaggctgatggagagtcgtgttcagcatctatgatgtaccaggaaggcaagttccgttatcggcgcgtggctgaaggcacccaggtgcttgagttgcccttcaaaggtgatgacatcaccatggtcctcatcttgcccaagcctgagaagagcctggccaaggtggagaaggaactcaccccagaggtgctgcaggagtggctggatgaattggaggagatgatgctggtggtccacatgccccgcttccgcattgaggacggcttcagtttgaaggagcagctgcaagacatgggccttgtcgatctgttcagccctgaaaagtccaaactcccaggtattgttgcagaaggccgagatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacatgcctttcctggtttttataagagaagttcctctgaacactattatcttcatgggcagggtagccaacccttgtgttaagtaaaatgttctctagaggatcccccatcgatggggtaccgagctcgaattc

SEQ ID NO: 4 - protein, 465 aa, Homo sapiens [sequence omitted]

SEQ ID NO: 5 - DNA, 988 bp, Homo sapiens
ggaacctctgcgagatttagaggaaagaaccagttttcaggcggattgcctcagatcacactatctccacttgcccagccctgtggaagattagcggccatgtattccaatgtgataggaactgtaacctctggaaaaaggaaggtttatcttttgtccttgctgctcattggcttctgggactgcgtgacctgtcacgggagccctgtggacatctgcacagccaagccgcgggacattcccatgaatcccatgtgcatttaccgctccccggagaagaaggcaactgaggatgagggctcagaacagaagatcccggaggccaccaaccggcgtgtctgggaactgtccaaggccaattcccgctttgctaccactttctatcagcacctggcagattccaagaatgacaatgataacattttcctgtcacccctgagtatctccacggcttttgctatgaccaagctgggtgcctgtaatgacaccctccagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacaggcctttcctggtttttataagagaagttcctctgaacactattatcttcatgggcagagtagccaacccttgtgttaagtaaaatgttcttattctttgcacctcttcctatttttggtttgtgaacagaagtaaaaataaatacaaactacttccatcgcaaaaaaaaaaaaaaaaaaaaaaaaaaaaaa

SEQ ID NO: 6 - protein, 259 aa, Homo sapiens
MYSNVIGTVTSGKRKVYLLSLLLIGFWDCVTCHGSPVDICTAKPRDIPMNPMCIYRSPEKKATEDEGSEQKIPEATNRRVWELSKANSRFATTFYQHLADSKNDNDNIFLSPLSISTAFAMTKLGACNDTLQQLMEVFKFDTISEKTSDQIHFFFAKLNCRLYRKANKSSKLVSANRLFGDKSLTFNDLYVSDAFHKAFLEVNEEGSEAAASTAVVIAGRSLNPNRVTFKANRPFLVFIREVPLNTIIFMGRVANPCVK

SEQ ID NO: 7 - DNA, 451 bp, Homo sapiens
cagcctagcttaacttggcattttgtctccttgcaggaaggtttatcttttgtccttgctgctcattggcttctgggactgcgtgacctgtcacgggagccctgtggacatctgcacagccaagccgcgggacattcacatgaatcccatgtgcatttaccgctccccggagaagaaggcaactgaggatgagggctcagaacagaagatcccggaggccaccaaccggcgtgtctgggaactgtccaaggccaattcccgctttgctaccactttctatcagcacctggcagattccaagaatgacaatgataacattttcctgtcacccctgagtatctccacggcttttgctatgaccaagctgggtgcctgtaatgacaccctccagcaactgatggaggtacgaccaaaggtcttctgcccagccaccttgttaggagcacctttg

SEQ ID NO: 8 - protein, 122 aa, Homo sapiens
KVYLLSLLLIGFWDCVTCHGSPVDICTAKPRDIHMNPMCIYRSPEKKATEDEGSEQKIPEATNRRVWELSKANSRFATTFYQHLADSKNDNDNIFLSPLSISTAFAMTKLGACNDTLQQLME

SEQ ID NO: 9 - DNA, 1751 bp, Ovis aries
ctcagaccacactatctccactcgctcagacctgtggaagattagtgaccatgatttccaatgggataggaaccgtcaccactgggaaaaggagcatgtgtcttttccctttgctgctcattggcctctggggctgtgtgacctgtcatcggagccctgtggaggacatctgcacagccaaacctcgggacattcctgtgaatcccatgtgtatttaccgttccccagagaagaaggcaactgagggagagggctcagagcagaagatccctggggccaccaaccggcgtgtctgggaactgtccaaggccaattcccactttgccactgccttctatcagcatttggcagactccaagaataacaatgacaacattttcctgtcacccctgagtatctccacagcttttgctatgaccaagctgggtgcctgtaacaacacactcaagcagttgatggaggtttttaagtttgataccatctctgagaaaacttctgatcagatccactttttctttgctaaactgaattgccgactctatcgaaaagccaataaatcctctgagttggtatcggccaaccgtctttttggagacaaatccattaccttcaatgagacctaccaggacatcagtgaggtggtatatggggccaagctccagcccctggacttcaagggaaatgcagagcagtccagattgactatcaaccaatggatatccaataagactgaagggcgtatcactgatgtcattcccccacaagccatcgatgagttcactgtcctggtgctagtcaacaccatttacttcaagggcctgtggaagtcaaagttcagtcccgagaacacaaagaaggagctgttctacaaggctgacggggagtcatgttcagtacccatgatgtaccaggaaggcaagttccgctatcggagagtggcagaaggcacccaggtgctcgagttgcccttcaagggtgatgacatcaccatggtgctcatcctgcccaagctggagaagcccctggccaaggtggaacgggagctcaccccagacatgctgcaggagtggctggatgagctgacagagacactgctggtggtccacatgccccacttccgcatcgaggacagcttcagcgtgaaggagcagctgcaagacatgggcctcgaagacttgttcagtcctgagaagtccaggctcccgggtattgttgcagaaggtcgaaacgacctctatgtctcagatgcattccacaaggcatttcttgaggtaaatgaggaaggcagtgaagctgcagcaagtaccgtcattagcatcgctggtcgttcgctgaacctgaacagggtgaccttccaggccaacaggcccttcctggttctcatcagggaagttgctctgaacactattatattcatgggcagagtagctaacccttgtgttaattaaaatgttatcctttgtatttcttcctattttggtttgtgaatacaagtaaaaataaatacaactattcccatatctgaccattatgaatggactctgccatctgaaatgaaggcaaggaaaggagaaatggatagagatgctgctgggcatttggtataacgaggctttcagcttttctctactggtgaacacatctgggtcaagaaagtgaaggagggagttatgttactacttcatcttgaaagatagtaggcatcttgagagggcagggtgagcttgaaatctagataatcccttaatact

SEQ ID NO: 10 - protein, 465 aa, Ovis aries
MISNGIGTVTTGKRSMCLFPLLLIGLWGCVTCHRSPVEDICTAKPRDIPVNPMCIYRSPEKKATEGEGSEQKIPGATNRRVWELSKANSHFATAFYQHLADSKNNNDNIFLSPLSISTAFAMTKLGACNNTLKQLMEVFKFDTISEKTSDQIHFFFAKLNCRLYRKANKSSELVSANRLFGDKSITFNETYQDISEVVYGAKLQPLDFKGNAEQSRLTINQWISNKTEGRITDVIPPQAIDEFTVLVLVNTIYFKGLWKSKFSPENTKKELFYKADGESCSVPMMYQEGKFRYRRVAEGTQVLELPFKGDDITMVLILPKLEKPLAKVERELTPDMLQEWLDELTETLLVVHMPHFRIEDSFSVKEQLQDMGLEDLFSPEKSRLPGIVAEGRNDLYVSDAFHKAFLEVNEEGSEAAASTVISIAGRSLNLNRVTFQANRPFLVLIREVALNTIIFMGRVANPCVN

SEQ ID NO: 11 - DNA, 1509 bp, Mus musculus
cttggagcatcggccatgtattcccctggggcaggaagtggggctgctggtgagaggaagctttgtctcctctctctgctcctcatcggtgccttgggctgtgctatctgtcacggaaaccctgtggacgacatctgcatagcgaagccccgagacatccccgtgaatcccttgtgcatttaccgctcccctgggaagaaggccaccgaggaggatggctcagagcagaaggttccagaagccaccaaccggcgggtctgggaactgtccaaggccaattcgcgatttgccactaacttctaccagcacctggcagactccaagaatgacaacgacaacattttcctgtcacccttgagcatctccactgcttttgctatgaccaagctgggtgcctgtaacgacactctcaagcagctgatggaggtttttaaatttgataccatctccgagaagacatccgaccagatccacttcttctttgccaaactgaactgccgactctatcgaaaagccaacaagtcctctgacttggtatcagccaaccgcctttttggagacaaatccctcaccttcaacgagagctatcaagatgttagtgaggttgtctatggagccaagctccagcccctggacttcaaggagaatccggagcaatccagagtgaccatcaacaactgggtagctaataagactgaaggccgcatcaaagatgtcatcccacagggcgccattaacgagctcactgccctggttctggttaacaccatttacttcaagggcctgtggaagtcaaagttcagccctgagaacacaaggaaggaaccgttctataaggtcgatgggcagtcatgcccagtgcctatgatgtaccaggaaggcaaattcaaataccggcgcgtggcagagggcacccaggtgctagagctgcccttcaagggggatgacatcaccatggtgctcatcctgcccaagcctgagaagagcctggccaaggtggagcaggagctcaccccagagctgctgcaggagtggctggatgagctgtcagagactatgcttgtggtccacatgccccgcttccgcaccgaggatggcttcagtctgaaggagcagctgcaagacatgggcctcattgatctcttcagccctgaaaagtcccaactcccagggatcgttgctggaggcagggacgacctctatgtctccgacgcattccacaaagcatttcttgaggtaaatgaggaaggcagtgaagcagcagcgagtacttctgtcgtgattactggccggtcactgaaccccaatagggtgaccttcaaggccaacaggcccttcctggttcttataagggaagttgcactgaacactattatattcatggggagagtggctaatccttgtgtgaactaaaatattcttaatctttgcaccttttcctactttggtgtttgtgaatagaagtaaaaataaatacgactgccacctcaaaaaaaaaaaaaaaaaaaa

SEQ ID NO: 12 - protein, 465 aa, Mus musculus
MYSPGAGSGAAGERKLCLLSLLLIGALGCAICHGNPVDDICIAKPRDIPVNPLCIYRSPGKKATEEDGSEQKVPEATNRRVWELSKANSRFATNFYQHLADSKNDNDNIFLSPLSISTAFAMTKLGACNDTLKQLMEVFKFDTISEKTSDQIHFFFAKLNCRLYRKANKSSDLVSANRLFGDKSLTFNESYQDVSEVVYGAKLQPLDFKENPEQSRVTINNWVANKTEGRIKDVIPQGAINELTALVLVNTIYFKGLWKSKFSPENTRKEPFYKVDGQSCPVPMMYQEGKFKYRRVAEGTQVLELPFKGDDITMVLILPKPEKSLAKVEQELTPELLQEWLDELSETMLVVHMPRFRTEDGFSLKEQLQDMGLIDLFSPEKSQLPGIVAGGRDDLYVSDAFHKAFLEVNEEGSEAAASTSVVITGRSLNPNRVTFKANRPFLVLIREVALNTIIFMGRVANPCVN

SEQ ID NO: 13 - DNA, 572 bp, Sus scrofa
ttcagagggattgcctcagaccacactatctccactcgcccagacctgtggaagattagcgaccatgttttccagtgggataggaactgtagctgctagaaaaaggagggagtgtcttctctccttgctgattattggcctctggggctgtgtgacctgtcattggagccctgtggaggacatctgcacagccaagcctcgggacattcccgtgaatcccatgtgcatttaccgttccccagagaagaaggccactgagggcgagggctcagagcagaagatccctgaggccaccaaccggcgggtctgggaactgtccaaggccaattcccactttgtcaccatcttctatcagcacttggcagactccaagaatgacaatgacaacattttcctgtcacccctgagtatctccacagcttttgctatgaccaagctgggtgcctgtgacaacaccctcaagcagctgatggaggtgtttaagtttgataccatctctgagaaaacatctgatcaggtccactttttctttgccaaactgaactgccgactctatcgaaaagccaacaagt

SEQ ID NO: 14 - protein, 431 aa, Sus scrofa
HWSPVEDICTAKPRDIPVNPMCIYRSPEKKATEGEGSEQKIPEATNRRVWELSKANSHFATIFYQHLADSKNDNDNIFLSPLSISTAFAMTKLGACDNTLKQLMEVFKFDTISEKTSDQVHFFFAKLNCRLYRKANKSSELVSANRLFGDKSLTFNETYQEISEVVYGAKLQPLDFKENAEQSRGIINQWVSNKTEGRITDVIPPEAINELTVLVLVNTIYFKGRWKSKFSSENTRKELFYKANGESCSVSMMYQESKFRYRRVAEGTQVLELPFKGDDITMVLILPKLEKSLAKVEQELTPEVLQEWLDELADTLLVVHMPRFRIEDSFSVKERLQDMGLEDLFIPEKAKLPGIVAEGRDDLYVSDAFHKAFLEVNEEGSEAAASTAIGIAGRSLNPARVTFKANRPFLVLIREVALNTIIFMGRVANPC

SEQ ID NO: 15 - DNA, 1260 bp, Rattus norvegicus
atgtgcatttaccgctcccctgcgaagaaggccacggaggaggatgtcctagagcagaaggttccggaagccaccaaccggcgggtctgggaactgtccaaggccaattctcgatttgccactaacttctatcagcacctggcagactccaagaacgacaacgacaacattttcctgtcacccttgagcatctccacggcgtttgctatgaccaagctgggtgcttgtaataacaccctcaagcagctgatggaggtttttaaatttgataccatctccgagaagacatccgaccagatccacttcttctttgccaaactgaactgccgactctatcgaaaagccaacaagtcctctaacttggtgtcagccaaccgcctttttggagacaaatcccttaccttcaatgagagctatcaagacgttagtgagattgtctatggagccaagcttcagcccctggacttcaaggagaatccggagcaatccagagtgaccatcaacaactgggtagctaataagactgaaggccgcatcaaagacgagaatccggagcaatccagagtgaccatcaacaactgggtagctaataagactgaaggccgcatcaaagacgtcatcccccaaggagccattgatgagctcactgccctggtgctggttaacaccatttacttcaagggcctgtggaagtcaaagttcagccctgagaacacaaggaaggaaccattccacaaagttgatgggcagtcatgcctggtgcccatgatgtaccaggaaggcaaattcaaatacaggcgtgtgggagagggtacccaggtgctagagatgcccttcaagggggacgacatcaccatggtgctcatcctgcccaagcctgagaagagcctggctaaggtggagcaggaactcaccccggagctgctgcaggagtggctggatgagctgtcggaggtcatgcttgtggtccacgtgccccgcttccgcatcgaggacagcttcagtctgaaggagcagctgcaagacatgggccttgttgatctcttcagccctgagaagtcccaactcccagcgtttcttgaggtaaatgaggaaggcagtgaagcagcagcgagtacttctgtcgtgattactggccggtcactgaaccccagtagggtgaccttcaaggccaacaggcccttcctggttcttataagggaagtcgcactgaacactattatattcatggggagagtgtctaatccttgtgtgaactaa

SEQ ID NO: 16 - protein, 419 aa, Rattus norvegicus
MCIYRSPAKKATEEDVLEQKVPEATNRRVWELSKANSRFATNFYQHLADSKNDNDNIFLSPLSISTAFAMTKLGACNNTLKQLMEVFKFDTISEKTSDQIHFFFAKLNCRLYRKANKSSNLVSANRLFGDKSLTFNESYQDVSEIVYGAKLQPLDFKENPEQSRVTINNWVANKTEGRIKDENPEQSRVTINNWVANKTEGRIKDVIPQGAIDELTALVLVNTIYFKGLWKSKFSPENTRKEPFHKVDGQSCLVPMMYQEGKFKYRRVGEGTQVLEMPFKGDDITMVLILPKPEKSLAKVEQELTPELLQEWLDELSEVMLVVHVPRFRIEDSFSLKEQLQDMGLVDLFSPEKSQLPAFLEVNEEGSEAAASTSVVITGRSLNPSRVTFKANRPFLVLIREVALNTIIFMGRVSNPCVN

SEQ ID NO: 17 - DNA, 1272 bp, Gallus gallus
cgggacatcccagtgaaccccatctgcatctaccgcaaccctgagaagaagccccaggaaaggcgaggtgctggagccggggaagggcaggatcccggagttcacaaacccccggtctgggagctgtccagggccaactcgcgtttcgccgtcgtcttctacaagcacctggccgactccaaggacaatgaggagaacatcttcctgtcgcccctcagcatttccacagcctttgccatgaccaagctcggggcgtgtggtgacaccctgcagcagctcatggaggtcttccagtttgacactatttcagagaagacatctgaccaggtccacttcttcttcgccaagctcaactgccgtctttacaagaaagccaacaagtcatcagagctaatatcagcaaaccgtctctttggagagaaatccttggtcttcaatgagacttaccagaacattagtgaaatagtttatggagccaaactctggccgttgaacttcaaagagaagccagagctttccaggaagatcataaacgagtgggtagccaataagacggagaggcgcattacagaagtgatcccagaaaaaggtatcgatgatctcactgtcttggtcctggtcaacaccatttattttaaggggcactggaagtcgcagttcccagctccaaacacgagactggatttatttcacaaagccaacggtgagacctgcaatgtccccatcatgtaccaggagtccaggttcccgtacgcgttcatccaggaggacaaagtccaggtgctggagctgccttacaaaggggacgacatcaccatggtgctggtcctgcccaaagctggcacaccgttggtggaggtggagcgagacctgacgtcggacaagctgcaagactggatcgactctatgatggaggtctcccttactgtctccttcccccgcttccgtgtcgaggacagcttcagtgtcaaggagaagctgagaaaaatggggctggaagatctcttcagtccagaaaatgccaagctgccaggtatagttgcaggggaccgcacagacctgtatgtatctgaggctttccacaaagccttccttgaggtgaatgaagaaggcagtgaggcgtcagcagcaacagctgttgttatctctggccgttccttccccatgaacagaattatctttgaagccaacaggcccttcttgctcttcatccgggaagccaccctcaacaccattatattcatgggcagaatatctgatccttgctcttaa

SEQ ID NO: 18 - protein, 423 aa, Gallus gallus
RDIPVNPICIYRNPEKKPQERRGAGAGEGQDPGVHKPPVWELSRANSRFAVVFYKHLADSKDNEENIFLSPLSISTAFAMTKLGACGDTLQQLMEVFQFDTISEKTSDQVHFFFAKLNCRLYKKANKSSELISANRLFGEKSLVFNETYQNISEIVYGAKLWPLNFKEKPELSRKIINEWVANKTERRITEVIPEKGIDDLTVLVLVNTIYFKGHWKSQFPAPNTRLDLFHKANGETCNVPIMYQESRFPYAFIQEDKVQVLELPYKGDDITMVLVLPKAGTPLVEVERDLTSDKLQDWIDSMMEVSLTVSFPRFRVEDSFSVKEKLRKMGLEDLFSPENAKLPGIVAGDRTDLYVSEAFHKAFLEVNEEGSEASAATAVVISGRSFPMNRIIFEANRPFLLFIREATLNTIIFMGRISDPCS

SEQ ID NO: 19 - DNA, 1371 bp, Xenopus laevis
atgtatctgctttcattgttgcttctcagcctcttgggctcagcatacctccagccacagcatgctgacatatgcctggcaaaacctaaagatatacctctgactcccatgtgtgtctaccggaaacctctggaggtggttgaaaccgaggaaaaggagaaagaacctacaacgcaagaacagaaggttcctgagtccactaaccctcgtgtatatgagctctcccaggccaatgctaaatttgcaattgctttctataaaaatctcgctgactccaagcgtgacaaagaaaatatctttatgtcacccctgagcatctctcaagcctttacaatggcaaaactgggtgcctgcaataacacactgaagcaacttatggaggtattccactttgacacagtttcagagcgggcttctgatcaaatacactacttctttgcaaagctcaactgccgcctgttcagaaaagcaaacaagtcatccgaactggtatctgtcaatcgcctttttggtgagaagtctctgacctttaatgaaacctatcaagatatcagtgagatagtgtatggggctaaattgtggcccttaaactttagggataagcctgaactatcccgtgaaataattaataattgggtatccaataaaacagagaagcgaataactgatgtgatccctaaggacgccatcactcctgacacagtattggtgctgataaatgccatctacttcaagggactttggaaatccaagtttaattcagaaaatacgaaaatggaccaattccacccagctaaaaattccaactgcttgactgcaaccatgtatcaagagggtacattccgttatggttcctttaaagatgatggagttcaggtccttgagctgccttataaaggtgatgacattacaatggtgctggtgctaccttcgcaagagactccgctaacaacagtggagcagaacctgacactggaaaagcttgggaattggctccagaagtctcgagaattacagttatctgtttatctccctcgattccgggtggaagattccttcagtgttaaggagaaattacaggaaatgggattggtagacctgtttgatccaaactcagcaaagcttccaggaatcattgcaggaggaaggacagacttgtatgtgtccgatgctttccataaggcatttttagaggtcaacgaggagggtagtgaggcagccgcatccactgccgtgattttgacaggacgttctttgaacctgaaccggatcatattcagagccaataggcccttcctggtctttatccgagaagttgctataaatgctattttgttcatggggagagtagctaacccctgcactgaatag

SEQ ID NO: 20 - protein, 456 aa, Xenopus laevis
MYLLSLLLLSLLGSAYLQPQHADICLAKPKDIPLTPMCVYRKPLEVVETEEKEKEPTTQEQKVPESTNPRVYELSQANAKFAIAFYKNLADSKRDKENIFMSPLSISQAFTMAKLGACNNTLKQLMEVFHFDTVSERASDQIHYFFAKLNCRLFRKANKSSELVSVNRLFGEKSLTFNETYQDISEIVYGAKLWPLNFRDKPELSREIINNWVSNKTEKRITDVIPKDAITPDTVLVLINAIYFKGLWKSKFNSENTKMDQFHPAKNSNCLTATMYQEGTFRYGSFKDDGVQVLELPYKGDDITMVLVLPSQETPLTTVEQNLTLEKLGNWLQKSRELQLSVYLPRFRVEDSFSVKEKLQEMGLVDLFDPNSAKLPGIIAGGRTDLYVSDAFHKAFLEVNEEGSEAAASTAVILTGRSLNLNRIIFRANRPFLVFIREVAINAILFMGRVANPCTE

SEQ ID NO: 21 - protein, 433 aa, Bos Taurus
HRSPVEDVCTAKPRDIPVNPMCIYRSSEKKATEGQGSEQKIPGATNRRVWELSKANSHFATAFYQHLADSKNNNDNIFLSPLSISTAFAMTKLGACNNTLTQLMEVFKFDTISEKTSDQIHFFFAKLNCRLYRKANKSSELVSANRLFGDKSITFNETYQDISEVVYGAKLQPLDFKGNAEQSRLTINQWISNKTEGRITDVIPPQAINEFTVLVLVNTIYFKGLWKSKFSPENTRKELFYKADGESCSVLMMYQESKFRYRRVAESTQVLELPFKGDDITMVLILPKLEKTLAKVEQELTPDMLQEWLDELTETLLVVHMPRFRIEDSFSVKEQLQDMGLEDLFSPEKSRLPGIVAEGRSDLYVSDAFHKAFLEVNEEGSEAAASTVISIAGRSLNSDRVTFKANRPFLVLIREVALNTIIFMGRVANPCVD

SEQ ID NO: 22 - protein, 6 aa, Artificial Sequence
  Description of Artificial Sequence Illustrative peptide
SEAAAS